SEQUENCE LISTING (15 sequences)

SEQ ID NO: 1 - protein, 20 aa, synthetic construct
IYQAGSTPCNGVEGFNCYFP

SEQ ID NO: 2 - protein, 20 aa, synthetic construct
LNEVAKNLNESLIDLQELGK

SEQ ID NO: 3 - protein, 4 aa, synthetic construct
GSSG

SEQ ID NO: 4 - protein, 152 aa, synthetic construct
AGNATEVPANSTVLSFCAFAVDPAKAYKDYLASGGQPITNCVKMLCTHTGTGQAITVTPEANMDQESFGGASCCLYCRCHIDHPNPKGFCDLKGKYVQIPTTCANDPVGFTLRNTVCTVCGMWKGYGCSCDQLREPLMQSADASTFLNGFAV

SEQ ID NO: 5 - protein, 267 aa, synthetic construct
MGIYQAGSTPCNGVEGFNCYFPGGSGIYQAGSTPCNGVEGFNCYFPGGSGAGNATEVPANSTVLSFCAFAVDPAKAYKDYLASGGQPITNCVKMLCTHTGTGQAITVTPEANMDQESFGGASCCLYCRCHIDHPNPKGFCDLKGKYVQIPTTCANDPVGFTLRNTVCTVCGMWKGYGCSCDQLREPLMQSADASTFLNGFAVGSSGLNEVAKNLNESLIDLQELGKGGSGLNEVAKNLNESLIDLQELGKGSENLYFQGSGHHHHHH

SEQ ID NO: 6 - protein, 162 aa, synthetic construct
IIKLLNEQVNKEMNSSNLYMSMSSWCYTHSLDGAGLFLFDHAAEEYEHAKKLIIFLNENNVPVQLTSISAPEHKFEGLTQIFQKAYEHEQHISESINNIVDHAIKSKDHATFNFLQWYVAEQHEEEVLFKDILDKIELIGNENHGLYLADQYVKGIAKSRKS

SEQ ID NO: 7 - protein, 252 aa, synthetic construct
MGIYQAGSTPCNGVEGFNCYFPGGSGIYQAGSTPCNGVEGFNCYFPGGSGIYQAGSTPCNGVEGFNCYFPGGSGIIKLLNEQVNKEMNSSNLYMSMSSWCYTHSLDGAGLFLFDHAAEEYEHAKKLIIFLNENNVPVQLTSISAPEHKFEGLTQIFQKAYEHEQHISESINNIVDHAIKSKDHATFNFLQWYVAEQHEEEVLFKDILDKIELIGNENHGLYLADQYVKGIAKSRKSGENLYFQGSGHHHHHH

SEQ ID NO: 8 - protein, 276 aa, synthetic construct
MGLNEVAKNLNESLIDLQELGKGGSGLNEVAKNLNESLIDLQELGKGGSGLNEVAKNLNESLIDLQELGKGGSGLNEVAKNLNESLIDLQELGKGGSGIIKLLNEQVNKEMNSSNLYMSMSSWCYTHSLDGAGLFLFDHAAEEYEHAKKLIIFLNENNVPVQLTSISAPEHKFEGLTQIFQKAYEHEQHISESINNIVDHAIKSKDHATFNFLQWYVAEQHEEEVLFKDILDKIELIGNENHGLYLADQYVKGIAKSRKSGENLYFQGSGHHHHHH

SEQ ID NO: 9 - protein, 250 aa, synthetic construct
MGIYQAGSTPCNGVEGFNCYFPGGSGIYQAGSTPCNGVEGFNCYFPGGSGAGNATEVPANSTVLSFCAFAVDPAKAYKDYLASGGQPITNCVKMLCTHTGTGQAITVTPEANMDQESFGGASCCLYCRCHIDHPNPKGFCDLKGKYVQIPTTCANDPVGFTLRNTVCTVCGMWKGYGCSCDQLREPLMQSADASTFLNGFAVGSSGLNEVAKNLNESLIDLQELGKGGSGLNEVAKNLNESLIDLQELGK

SEQ ID NO: 10 - protein, 346 aa, synthetic construct
MGIYQAGSKPCNGVEGFNCYFPGGSGYRYRLFRKSNLKPFERDISTGGSGIYQAGSKPCNGVEGFNCYFPGGSGYRYRLFRKSNLKPFERDISTGGSGAGNATEVPANSTVLSFCAFAVDPAKAYKDYLASGGQPITNCVKMLCTHTGTGQAITVTPEANMDQESFGGASCCLYCRCHIDHPNPKGFCDLKGKYVQIPTTCANDPVGFTLRNTVCTVCGMWKGYGCSCDQLREPLMQSADASTFLNGFAVGSSGKPSKRSFIEDLLFNKVTLADGGSGLNEVAKNLNESLIDLQELGKGGSGKPSKRSFIEDLLFNKVTLADGGSGLNEVAKNLNESLIDLQELGK

SEQ ID NO: 11 - protein, 570 aa, synthetic construct
MGRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTYGVGYQPYRVVVLSFELLHAPATVCGPKKSTGGSGAGNATEVPANSTVLSFCAFAVDPAKAYKDYLASGGQPITNCVKMLCTHTGTGQAITVTPEANMDQESFGGASCCLYCRCHIDHPNPKGFCDLKGKYVQIPTTCANDPVGFTLRNTVCTVCGMWKGYGCSCDQLREPLMQSADASTFLNGFAVGGSGRFPNITNLCPFDEVFNATRFASVYAWNRKRISNCVADYSVLYNLAPFFTFKCYGVSPTKLNDLCFTNVYADSFVIRGDEVRQIAPGQTGNIADYNYKLPDDFTGCVIAWNSNKLDSKVSGNYNYLYRLFRKSNLKPFERDISTEIYQAGNKPCNGVAGFNCYFPLKSYSFRPTYGVGHQPYRVVVLSFELLHAPATVCGPKKST

SEQ ID NO: 12 - protein, 362 aa, synthetic construct
MGVSIVPSTVWEDGDYYRKQLSGGSGWPLNDYGFYTTTGIGYQPYRGGSGFPLQSYGFQPTNGVGYQPYRGGSGFPLKSYSFRPTYGVGHQPYRGGSGAGNATEVPANSTVLSFCAFAVDPAKAYKDYLASGGQPITNCVKMLCTHTGTGQAITVTPEANMDQESFGGASCCLYCRCHIDHPNPKGFCDLKGKYVQIPTTCANDPVGFTLRNTVCTVCGMWKGYGCSCDQLREPLMQSADASTFLNGFAVGSSGKPSKRSFIEDLLFNKVTLADGSSGSRSARSAIEDLLFDKVTIADGSSGDSFKEELDKYFKNHTSPDVDGSSGFQDELDEFFKNVSTSIPNFGGENLYFQGSGHHHHHH

SEQ ID NO: 13 - protein, 312 aa, synthetic construct
MGGDILSRLDKVEAEVQIDRLITGRLQSLQTFVGGSGGDIIQRLDVLEQDAQIDRLINGRLTTLNAFVGGSGAGNATEVPANSTVLSFCAFAVDPAKAYKDYLASGGQPITNCVKMLCTHTGTGQAITVTPEANMDQESFGGASCCLYCRCHIDHPNPKGFCDLKGKYVQIPTTCANDPVGFTLRNTVCTVCGMWKGYGCSCDQLREPLMQSADASTFLNGFAVGSSGKPSKRSFIEDLLFNKVTLADGSSGSRSARSAIEDLLFDKVTIADGSSGKPSKRSFIEDLLFNKVTLADGENLYFQGSGHHHHHH

SEQ ID NO: 14 - protein, 876 aa, synthetic construct
MGKTISVVTLLCVLPAVVYSTCTVPTMNNAKLTSTETSFNDKQKVTFTCDSGYHSLDPNAVCETDKWKYENPCKKMCTVSDYVSELYDKPLYEVNSTMTLSCNGETKYFRCEEKNGNTSWNDTVTCPNAECQPLQLEHGSCQPVKEKYSFGEYMTINCDVGYEVIGVSYISCTANSWNVIPSCQQKCDIPSLSNGLISGSTFSIGGVIHLSCKSGFTLTGSPSSTCIDGKWNPILPTCVRSNEEFDPVDDGPDDETDLSKLSKDVVQYEQEIESLEATYHGGSGGGSGDGTLFPGDDDLAIPATEFFSTKAAKNPETKREAIVKAYGDDNEETLKQRLTNLEKKITNITTKFEQIEKCCKRNDEVLFRLENHAETLRAAMISLAKKIDVQTGRHPYEGGSGGGSGAGNATEVPANSTVLSFCAFAVDPAKAYKDYLASGGQPITNCVKMLCTHTGTGQAITVTPEANMDQESFGGASCCLYCRCHIDHPNPKGFCDLKGKYVQIPTTCANDPVGFTLRNTVCTVCGMWKGYGCSCDQLREPLMQSADASTFLNGFAVGGSGGGSGSIQTTVNTLSERISSKLEQEANASAQTKCDIEIGNFYIRQNHGCNITVKNMCSADADAQLDAVLSAATETYSGLTPEQKAYVPAMFTAALNIQTSVNTVVRDFENYVKQTCNSSAVVDNKLKIQNVIIDECYGAPGSPTNLEFINTGSSKGNCAIKALMQLTTKATTQIAPRQVAGTGVQFGGSGGGSGNQCMSANKAAITDSAVAVAAASSTHRKVVSSTTQYDHKESCNGLYYQGSCYILHSDYKSFEDAKANCAAESSTLPNKSDVLTTWLIDYVEDTWGSDGNPITKTTSDYQDSDVSQEVRKYFCT

SEQ ID NO: 15 - protein, 324 aa, synthetic construct
MGNRDFVEGVSGGSWVDIVLGGSGGLFGKGGIVTCAMFTCKKGGSGVLLQMEEKAWLVHRQWFLGGSGAGNATEVPANSTVLSFCAFAVDPAKAYKDYLASGGQPITNCVKMLCTHTGTGQAITVTPEANMDQESFGGASCCLYCRCHIDHPNPKGFCDLKGKYVQIPTTCANDPVGFTLRNTVCTVCGMWKGYGCSCDQLREPLMQSADASTFLNGFAVGGSGMHTALTGATEIQMSSGNLGGSGVKEIAETQHGTIVIRVQYGGSGAIYGAAFSGVSWTMKILIGGSGGKVVLPENLEYTIVITPHGENLYFQGSGHHHHHH

(19) United States
(12) Patent Application Publication
(10) Pub. No.: US 2024/0424087 A1
(43) Pub. Date: Dec. 26, 2024

(54) MULTIEPITOPE SELF-ASSEMBLED NANOPARTICLE VACCINE PLATFORM (MSN-VACCINE PLATFORM) AND USES THERE OF

(71) Applicant: Translational Health Science and Technology Institute, Haryana faridabad (IN)

(72) Inventors: Sweety Samal, Haryana faridabad (IN); Shubbir Ahmed, Haryana faridabad (IN); Amit Awasthi, Haryana faridabad (IN); Ritika Khatri, Haryana faridabad (IN)

(21) Appl. No.: 18/700,563

(22) PCT Filed: Oct. 11, 2022
(86) PCT No.: PCT/IB22/59713

§ 371 Date: Apr. 11, 2024

(30) Foreign Application Priority Data
Oct. 11, 2021 (IN) 202111046243

(51) Int. Cl. A61K 39/215; A61K 39/00; A61P 31/14; C12N 7/00

(52) U.S. Cl. CPC A61K 39/215; A61P 31/14; C12N 7/00; A61K 2039/575; A61K 2039/70; C12N 2710/24134; C12N 2770/20023; C12N 2770/20033; C12N 2770/24134

(57) ABSTRACT

The present invention is drawn to a next generation nano vaccine platform by using structure-based design to utilize the conserved or less variable or highly immunogenic domains or epitopes and displaying it in a nano cage and produces it in as nanoparticle protein in prokaryotic expression system. The present invention is illustrated in detail by a vaccine design and construct for SARS CoV-2, SARS-CoV-2 variants, betacorona viruses, Monkey pox virus and Dengue virus.

### FIELD OF THE INVENTION

The present invention relates to development of Multivalent Self-assembled Nanoparticle vaccine platform (MSN-vaccine platform) and uses thereof.

### BACKGROUND OF THE INVENTION

Vaccines are the most effective health interventions. They protect us from contagious diseases for examples hepatitis B, influenza, small pox, polio. With the emergence of SARS-CoV-2 and its' pandemic status, immediate and effective medical intervention is urgently required for important pathogens with pandemic potential.

For enveloped viruses the vaccine is mostly targeted to the structural envelope protein which is highly immunogenic and provides protection. One of the approaches for development of vaccine targeted to the structural envelope is production of soluble recombinant protein. However, design and development of soluble protein subunit-based vaccine candidate for viral pathogen is challenging. The viral proteins are large, complex and have post translational modifications such as glycosylation. The viral pathogens use complex protein synthesis system of their eukaryotic host such as mammal. However, recombinant protein production in mammalian system incurs high cost due to low yield, costly reagents and complex downstream processing. Besides, in RNA viruses, the structural envelope proteins continue to evolve and mutate. In addition, the high production cost in mammalian expression system makes vaccine availability difficult to LMICs. Contrary, the prokaryotic system is cost effective because of high yield and cheap upstream and downstream stages. However, enveloped subunit protein-based vaccine production in prokaryotic expression system has not been successful so far as the viral protein produced in prokaryotic system form non-native-like structure and often form large protein aggregates or expressed in low quantity or found in inclusion bodies. Based on proteins, it is very difficult to develop multivalent vaccines with broad efficacy.

Furthermore, besides the protein-based vaccine, the traditional vaccines also are linked to several disadvantages; such as induction of allergy and autoimmune reactions, have low stability and the recently emerged new vaccine technology like mRNA vaccines need storage at a cold temperature which is not very feasible for tropical countries like India.

Both RNA and DNA viruses cause harmful diseases in animals and humans and in many cases they have zoonotic importance with pandemic potential, as seen in recently emerged viruses—severe acute respiratory syndrome coronavirus 2 (SARS-COV-2, RNA virus) virus and Monkey pox virus (DNA virus).

SARS-COV-2 belongs to coronaviruses whose genomes encode for four or five structural proteins known as spike(S), membrane (M), envelope (E), nucleocapsid (N) (Liang et al., 2020 Front. Immunol. 11, 1022.). The mature virions mainly contain the viral structural proteins whereas non-structural proteins, encoded and expressed in infected cells and are not assembled in the virion surface (Lazarowitz et al., 1971 Virology 46 (3), 830-843.). The spike(S) of SARS-COV-2 also resembles in its structural organization and functions to other class 1 viral envelope and fusion proteins, such as HIV-1 envelop gp160, HCV, chikungunya, HA protein from influenza virus, and Ebola GP protein (Banerjee N et.al, Virus Disease. (2016) 27:1-11) and S protein-based subunit vaccines have shown to induce immune responses (Chen.et.al 2020, Curr Trop Med Reports. (2020) 7:61-4, Jiang S el.al, 2012 Expert Rev Vaccines. (2012) 11:1405-13, Smith TRF et.al Nat Commun. (2020) 11:2601, Ravichandran S et.al, Sci Transl Med. (2020) 12:1-9). Spike of SARS-COV-2 consists of the S1 major domain which can be divided into four sub-domains: NTD (N-terminal domain), receptor binding domain (RBD), and two CTDs (C-terminal domains). The S2-protein domain, consists of a second proteolytic site (S2′) upstream of the fusion peptide (FP), an internal fusion peptide (IFP), that is similar in SARS-COV and SARS-COV-2, and along with that two heptad-repeat domains preceding the transmembrane domain (TM) (Wang D. et al., 2020 Vaccines 8 (3), 355.). SARS-COV-2 RBD attaches to angiotensin-converting enzyme 2 (ACE2) receptor and facilitates virus entry (Li, 2013 Antivir. Res. 100 (1), 246-254.; Walls A C et.al, Cell. (2020) 181:281-92.e6., hence antibodies against spike protein can block virus entry, thus S or RBD based immunogens are excellent vaccine candidates. The spike protein is a homotrimeric protein on the virion surface. Even though the whole trimeric S protein or monomeric spike or RBD protein arepotential vaccine candidates; however, the viruses from this family continuously mutate and new variants produce which affects the vaccine efficacy (Salvatori G et.al J Transl Med. (2020) 18:1-3., Weisblum Y et.al Elife. 2020 Oct. 28; 9: e61312., Philip R et.al, N Engl J Med 2021; 385:179-186, Alter G et.al Nature. 2021 Jun. 9. doi: 10.1038/s41586-021-03681-2., Takuya Tada et.al, bioRxiv 2021 Jul. 21;2021.07.19.452771, Krause P R et.al N Engl J Med. 2021 Jul. 8; 385 (2): 179-186). In addition, it has been seen that in some cases, the full-length S protein induces enhanced infectivity and infiltration of eosinophils which might be due to antibody-dependent enhancement (ADE) effect (Padron-Regalado E et.al Infect Dis Ther. (2020) 9:255-74., Luo F et.al Virol Sin. (2018) 33:201-4, Jaume M et.al J Virol. (2011) 85:10582-97). SARS-COV-2. Spike protein consists of many immunodominant domains those could elicit strong antibody responses. Berry J D et.al MAbs. (2010) 2:53-66., have shown the neutralizing epitopes present in the SARS-COV spike protein. It was shown by Ortega et al. EXCLI J. 2020, 19, 410-417, in SARS-COV S1 a small fragment of 18 amino acids located into RBD (473-N to 491-Y) is responsible for the recognition of hACE 2 and from these 18 amino acids upto eight amino acids are also preserved in SARS-COV-2. In addition, S2induced higher antibody binding have been found in COVID-19 patients with respect to those against the entire RBD and S1, suggesting that S2 is more exposed as compared to other regions (Dai L, et al. Nat Rev Immunol. 2021 21(2):73-82.).

Dengue is another important enveloped RNA virus and presently there is only one approved and licensed vaccine against Dengue virus (DENV), Dengvaxia® (CYD-TDV), developed by Sanofi Pasteur. DENV has four serotypes and one the major challenge in vaccine development is induction of antibody dependent enhancement of disease due to preexisting immunity against DENV serotypes and high cross reactivity.

Monkey pox is an important zoonotic disease, which has re-emerged in recent times. It caused by double stranded DNA monkey pox virus that belongs to the genus Orthopoxvirus, family Poxviridae, and sub-family Chordopoxvirinae. There are two vaccines that are currently approved in and provide protection against monkey pox. The Jynneos vaccine contains live attenuated vaccinia virus and could be used for the prevention of smallpox and monkeypox disease in adults ages 18 and older who are at high risk for infection with either virus. The ACAM2000, FDA licensed Smallpox (Vaccinia) Vaccine and is for active immunization against smallpox disease for persons who are at high risk for smallpox infection. Even though, it is believed that both the vaccines could provide protection against the monkey pox infection, however the virus is emerging and new non-immune generations might contribute to increase in the incidence.

Vaccines mainly target the most immunogenic part of the virus and in case of enveloped virus, this is mainly the envelope protein that protrudes from the virus surface. As discussed earlier, different approaches taken for vaccine development and each platform or approach has advantages and limitations. For example, current SARS-COV-2 vaccines are mainly based on four promising vaccine platforms i) mRNA ii) inactivated virus iii) subunit protein iv) live viral vector. In these vaccine platforms, the target is one or two of most immunogenic parts of the same virus to elicit protective antibodies.

However, RNA viruses tend to continuously evolve with emergence of new strains. Highly immunogenic parts are also prone to these evolutionary dynamics that may result in lower efficacy of vaccines and eventually, give rise to a new dominant virus or strain with immune escape. A similar phenomenon is being observed in the ongoing SARS-COV-2 pandemic. With the emergence of recent SARS-COV-2 variants which are more infectious, high transmissibility and associated with more disease severity, it is important to incorporate innovative platform and newer technology for the development of next generation vaccine platform by improving vaccine candidate that could provide heterogenic and broader immunity and which remains a public priority. The emergence of new variants and presence of large hosts beyond bats, coronaviruses infect camels, birds, cats, horses, mink, pigs, rabbits, pangolins, and other animals, the threat for emergence of a new pandemic seems real.

A pan-corona vaccine or mosaic vaccine that can protect against more than one variant, which can be rapidly and easily manufactured is the most promising strategy that holds the solution to future pandemic. Additionally, for the recently emerged Monkey pox virus, there are more than one antigen that is protective, hence there is a need to develop a vaccine incorporating all the main protective antigens into the vaccine.

Peptide-or small epitope based immunogens are smart approaches (Di Natale C, et al. Front Pharmacol. 2020. Dec. 3; 11:578382) that are able to overcome disadvantages of other vaccine design and development strategy as they are safe, easy to purify (Marasco et al., 2008 Curr. Protein Pept. Sci. 9 (5), 447-467; Marasco and Scognamiglio, 2015 Int. J. Mol. Sci. 16 (4), 7394-7412), the chemical synthesis makes them stable for range of storage temperature and rendering high reproducibility (Tizzano et al., 2005 Proteins 59 (1), 72-79.). However, inside body they are poorly represented to antigen presenting cells (APC) and easily degraded by proteases before eliciting an efficient immune response. In addition, standalone peptides are weak immunogens and they need adjuvants which act as additional immune stimulants to induce an effective immune response. The immunogenic peptides or epitopes showing desired antigenic or immunogenic properties, can be exploited in specific delivery carriers such as virus like particles (VLPs), liposomes, polymeric micro-/nano-particles for better representation to the immune cells for induction of suitable protective immune responses (Neek et al., 2019 Nanomed. Nanotechnol. Biol. Med. 15 (1), 164-174; Singh et al., 2007 Expert Rev. Vaccine 6 (5), 797-808). In addition, if multi-copies of antigenic peptides or epitopes or domains are added, it enhances immunogenic response and stabilize in a structural conformation to protect from protease degradation and can be used for ease of delivery (Joshi et al., 2013 Indian J. Virol. 24 (3), 312-320). The multiple copies could be 1) homologous that contains multimers of the same peptide or domains or epitopes 2) heterologous obtained by the combination of different epitopes or domains from different virus strains or different viruses from same family. For example, classical swine fever virus (CSFV) vaccine candidate B4T-type vaccine contains one T-cell epitope from the NS2-3 protein which also linked to four copies of B-cell epitopes from the E2 protein (Monso et al., 2011 J. Pept. Sci. 17 (1), 24-31; Joshi et al., 2013 Indian J. Virol. 24 (3), 312-320).

Accordingly, a vaccine platform that can incorporate highly immunogenic or conserved epitopes or domains or antigens in multiple copies in one unit and can be expressed as recombinant soluble protein is highly desirable and promising for the development of sub unit protein-based vaccines.

Further, most of the vaccines currently available are only amenable to mammalian expression systems, have a very low protein yield, cost of production is high, not secreted in the medium as soluble proteins and generally present in inclusion bodies, therefore increasing the steps in downstream processing and therefore very difficult to scale up.

Hence, there is a need for a vaccine technology that can overcome the problems of the prior art which could be easily producible, ensure scale up and affordability.

### OBJECT OF THE INVENTION

An object of the invention is to provide a vaccine platform to overcome the limitations of the current technologies including antigenic variation, scaling, and speed to the market and providing a next-generation self-assembled nanoparticle-based vaccine platform.

It is another object of the present invention to provide a method for production of next-generation self-assembled nanoparticle-based vaccine platform.

It is further an object of the present invention to provide a vaccine platform by using a conserved, less variable, immunodominant peptides, epitopes domains.

It is another object of the present invention to provide a prokaryotic expression platform to ensure easy scale-up and production with affordability.

### BRIEF DESCRIPTION OF THE FIGURES

FIG. 1 depicts the schematic of development of Multivalent Self-assembled Nanoparticle vaccine platform (MSN-vaccine platform), A. Enveloped protein B. identification or selection of antigenic peptide or epitope C. Screening of antigenic peptides and down selection of peptides showing high immunogenicity D. stitching of peptides to form multi-peptide or multiepitope by linker. E. Stapling of multiepitopes to suitable nano cage by linker F. Development of expression vector consisting of codon optimized polypeptide G. Downstream process development of expression, purification of nano particle based subunit soluble immunogen.

FIG. 2 depicts design of Nano A. (A) Stapled SARS-COV-2 spike protein 20 mer RBD peptide and conserved 20 mer peptides from S2 domain (conserved heptad repeat 2) by linkers. (B) depicts plasmid map (Expression Vector) of Nano A.

FIG. 3 depicts the schematic of design and plasmid map of Nano B and Nano C.

FIG. 4 depicts schematic of tagless construct.

FIG. 5 depicts the expression of Nano A, B, C in E. coli in pellet (insoluble fraction) and supernatant (soluble fraction)

FIG. 6 depicts the purified proteins Nano A and Nano C.

FIG. 7 depicts Western blot analysis of Nano A, B, C probed with anti-His commercial antibody.

FIG. 8 depicts native page analysis of A. Nano A B. Nano C nano protein.

FIG. 9 depicts Antigenicity of Nano A.

FIG. 10 depicts Size exclusion chromatography purification of Nano A and subsequent biochemical analysis.

FIG. 11 depicts the secondary structure signature of Nano A (A) and Nano C (B) as measured by using circular dichroism (CD) spectroscopy.

FIG. 12 depicts tag free purification of Nano A in presence of TEV protease and subsequent western blot analysis.

FIG. 13 depicts schematic of immunogenicity assessment of tagless Nano A protein immunogen (also termed as DS1) in BALB/c mouse.

FIG. 14 depicts humoral responses as measured by ELISA

FIG. 15 depicts the neutralization titer of immunized boost sera of tagless Nano A (DS1) against SARS-COV-2 Wu-1 live virus as measured by plaque neutralization assay.

FIG. 16 depicts designing of DS2.

FIG. 17 depicts the antigenic characterization of DS2.

FIG. 18 depicts the immunogenicity assessment of the immunogen DS2 in BALB/c mouse.

FIG. 19 depicts A. designing and 3D model of the immunogen DS3 and. B. represents the expression of the DS3 antigen.

FIG. 20 depicts the bioinformatic analysis of SARS-COV-1, CoV-2 and MERS spike domains for designing of betacorona vaccine.

FIG. 21 depicts A. the designing of the immunogen Pan-N3 and B represents the antigenic characterization.

FIG. 22 depicts the immunogenicity assessment of the immunogen pan-N3 in BALB/c mouse.

FIG. 23 depicts A. the designing of the immunogen pan-N4, and B. represents the antigenic characterization of the immunogen pan-N4.

FIG. 24 depicts the immunogenicity assessment of the immunogen pan-N4 in BALB/c mouse

FIG. 25 depicts the designing of subunit immunogen against the emerging Monkey pox virus.

FIG. 26 depicts the designing of subunit immunogen against the Dengue virus (DENV).

### SUMMARY OF THE INVENTION

In one aspect, the present invention provides a next generation multivalent nano vaccine platform that employs structure-based design to utilize the conserved or less variable domains or highly immunogenic epitopes or domains and displaying it in as nanoparticle protein in expression systems including prokaryotic expression system, mammalian expression system, yeast expression system or insect expression system.

The present invention is illustrated in detail for a vaccine design and constructs for SARS COV-2 and pan betacorona virus which includes viruses from SARS-COV-1, MERS and SARS-COV-2 and other variants of concern. Further enhancement is done in designing the antigen constructs for Dengue (by stapling the epitopes from the envelop protein) and Monkey pox (by stapling the antigens from intracellular mature virions-IMV and extra cellular virion-EEV) for vaccines development.

In an aspect of the present invention there is provided a method of designing a multivalent self-assembled nanoparticle vaccine comprising the steps of:
i. identifying an antigenic peptide or peptide selected from one or more immunogenic peptides, epitopes, domains from the conserved region of a virus or different strains of a virus of the same family;
ii. screening one or more antigenic peptide, immunogenic peptides, epitopes or domains identified in step (i) and selection of at least one immunogenic peptide or epitope or domain or antigenic peptide showing high immunogenicity;
iii. stapling said one or more antigenic peptide, immunogenic peptide, epitope or domain screened in step (ii) by one or more linker;
iv. stitching said stapled one or more antigenic peptide, immunogenic peptide, epitope or domain of step (iii) to a nano-cage by one or more linker to obtain a multipeptide;
v. optimizing codons for expressing said stitched multipeptide to obtain an encoding polynucleotide;
vi. developing an expression vector comprising said encoding polynucleotide;
vii. expressing said vector in a cell to obtain a multivalent self-assembled nanoparticle vaccine.

In a further step, the method may involve antigenic characterization and immunogenicity assessment of the multivalent self-assembled nanoparticle vaccine by using suitable adjuvant to induce antibody responses.

In an aspect of the invention there is provided a multivalent self-assembled nanoparticle vaccine comprising i. a multi-peptides comprising one or more immunogenic peptides stitched by one or more linker; and ii. a nanocage, wherein the multi-peptide is stapled with the nanocage.

In an aspect of the present invention there is provided an engineered SARS-COV-2 multipeptide having SEQ ID: 5, comprising at least two SARS-COV-2 receptor binding domain (RBD) peptides of SEQ ID: 1 and at least two peptides from heptad repeat (2) region from spike protein of SEQ ID: 2 linked by linker of SEQ ID: 3, and nanocage of SEQ ID: 4.

In an aspect there is provided a polynucleotide encoding the SARS-COV-2 multipeptide having SEQ ID: 5.

In an aspect there is provided an engineered SARS-COV-2 multipeptide having SEQ ID: 7, comprising at least two SARS-COV-2 receptor binding domain (RBD) peptides of SEQ ID: 1 linked by linker of SEQ ID: 3, and nanocage of SEQ ID: 6.

In an aspect there is provided a polynucleotide encoding the SARS-COV-2 multipeptide having SEQ ID: 7.

In an aspect there is provided an engineered SARS-COV-2 multipeptide having SEQ ID: 8, comprising at least two peptides from heptad repeat (2) region from spike protein of SEQ ID: 2 linked by linker of SEQ ID: 3, and nanocage of SEQ ID: 6.

In an aspect there is provided a polynucleotide encoding the SARS-COV-2 multipeptide having SEQ ID: 8.

In a further aspect there is provided a multivalent self-assembled nanoparticle vaccine having SEQ ID: 9.

In an aspect there is provided the complete sequence of the immunogen DS2 which consists of epitopes from SARS-COV-2 ancestral Wu-1 and epitopes from the SARS-COV-2 Delta variant, termed as SEQ ID 10.

In an aspect there is provided the complete sequence of the immunogen DS3 which consists of the RBD domain from SARS-COV-2 variant Alpha in the N-terminal domain of NSP-10 and RBD domain from the SARS-COV-2 variant Omicron in the C terminal domain of NSP-10 termed as SEQ ID 11.

In an aspect there is provided the complete sequence of the immunogen for pan beta corona vaccinepan-N3 which consists of the epitopes from SARS-COV-2, SARS-COV-1 and MERS spike domains termed as SEQ ID 12.

In an aspect there is provided the complete sequence of the immunogen for pan beta corona vaccine pan-N4 which consists of the conserved epitopes from SARS-CoV-2, SARS-COV-1 and MERS spike domains termed as SEQ ID 13.

In an aspect there is provided the complete sequence of the immunogen for Monkey pox virus vaccine MPXV-1 which consists of the antigens from both IMV and EEV particles termed as SEQ 1D 14.

In an aspect there is provided the complete sequence of the immunogen for Dengue virus vaccine DN-1 which consists of the conserved epitopes from DENV2 serotypes termed as SEQ 1D 15.

### DETAILED DESCRIPTION OF THE INVENTION

The present invention is related to the conventional techniques of molecular biology, microbiology, immunology, and vaccination.

The following description with reference to the accompanying drawings is provided to assist in a comprehensive understanding of various embodiments of the present disclosure as defined by the claims and their equivalents. It includes various specific details to assist in that understanding but these are to be regarded as merely exemplary. Accordingly, those of skill in the art will recognize that various changes and modifications of the various embodiments described herein can be made without departing from the scope and spirit of the present disclosure. In addition, descriptions of well-known functions and constructions may be omitted for clarity and conciseness.

It is to be understood that the singular forms “a”, “an”, and “the” include plural referents unless the context clearly dictates otherwise. Thus, for example, reference to “a component surface” includes reference to one or more of such surfaces.

The terminology used herein is for the purpose of describing particular various embodiments only and is not intended to be limiting of various embodiments. It will be further understood that the terms “comprises” and/or “comprising” used herein specify the presence of stated features, integers, steps, operations, members, components, and/or groups thereof, but do not preclude the presence or addition of one or more other features, integers, steps, operations, members, components, and/or groups thereof. Also, expressions such as “at least one of” when preceding a list of elements indicates that either one element or more than one of such elements can be used in combination.

The present invention is directed to a multivalent self-assembled nanoparticle vaccine and method of designing and development thereof.

The term “epitopes,” as used herein, refers to portions of a polypeptide having antigenic or immunogenic activity in an animal, for example a mammal, for example, a human. An “immunogenic epitope,” as used herein, is defined as a portion of a protein that elicits an immune response in an animal, as determined by any method known in the art. The term “antigenic epitope,” as used herein, is defined as a portion of a protein to which an antibody or T-cell receptor can immunospecifically bind its antigen as determined by any method well known in the art. Immunospecific binding excludes non-specific binding but does not necessarily exclude cross-reactivity with other antigens. Whereas all immunogenic epitopes are antigenic, antigenic epitopes need not be immunogenic.

The present invention utilizes a unique design approach to exploit the antigenic peptides of viral structural proteins with the potential to induce humoral and cellular immunity. The peptide-based vaccine of the present invention is easy to produce and amenable to tweaking for different serotypes or strains of viruses. Selecting conserved immunogenic peptides across different strain of a virus has the potential to induce broader protection. This is of particular importance in case of viral vaccines, such as SARS and related RNA viruses, where a broad vaccine candidate will not only give protection to the emerging variants but designed suitably may give protection for the new emerging variants. The antigenic peptides alone are less immunogenic and have low bioavailability. In addition, vaccine efficacy does not only depend on the biochemical composition of the immunogens but also their morphological properties, such as particle size, and if the vaccines delivered in the form of particulate are likely to be uptake by APCs for immune processing.

As used herein, the term “peptide” refers to a molecular chain of amino acids, which, if required, can be modified in vivo or in vitro, for example by manosylation, glycosylation, carboxylation or phosphorylation with the stipulation that these modifications must preserve the biological activity of the original molecule. In addition, peptides are referred to “epitopes” and part of protein. Functional derivatives of the peptides are also included in the present invention. Functional derivatives are meant to include peptides which differ in one or more amino acids in the overall sequence, which have deletions, substitutions, inversions or additions. Amino acid substitutions which can be expected not to essentially alter biological and immunological activities have been described. The peptides according to the invention can be produced synthetically, by recombinant DNA technology. Methods for producing synthetic peptides are well known in the art.

As used herein, the term “polypeptide” is intended to encompass a singular “polypeptide” as well as plural “polypeptides,” and comprises any chain or chains of two or more amino acids. Thus, as used herein, terms including, but not limited to “peptide,” “dipeptide,” “tripeptide,” “protein,” “amino acid chain,” or any other term used to refer to a chain or chains of two or more amino acids, are included in the definition of a “polypeptide,” and the term “polypeptide” may be used instead of, or interchangeably with any of these terms.

“Codon-optimization” is defined herein as modifying a nucleic acid sequence for enhanced expression in a specified host cell by replacing at least one, more than one, or a significant number, of codons of the native sequence with codons that are more frequently or most frequently used in the genes of that host. Various species exhibit particular bias for certain codons of a particular amino acid.

“Vaccine” as used herein is a composition comprising an immunogenic agent and a pharmaceutically acceptable diluent in combination with excipient, adjuvant, additive and/or protectant. The immunogen may be comprised of recombinantly produced soluble immunogens in form of nano particle based soluble protein including without limitation, polypeptides or polynucleotides). For example, a “Nano A vaccine” as used herein means a composition comprising an isolated multi epitope or multi peptide based nanoparticle comprising at least one polynucleotide encoding multiple copies of immunogenic peptides (e.g., SARS-CoV-2 receptor binding domain peptides) and a pharmaceutically acceptable diluent in combination with excipient, adjuvant, additive and/or protectant. When the vaccine is administered to a subject, the immunogen stimulates an immune response that upon subsequent challenge with infectious agent, protect the subject from illness or mitigate the pathology, symptoms or clinical manifestations caused by that agent. The vaccine, according to the invention, can be either therapeutic or prophylactic. A therapeutic (treatment) vaccine is given after infection and is intended to reduce or arrest disease progression. A preventive (prophylactic) vaccine is intended to prevent initial infection or reduce the burden of the infection. A vaccine may further comprise other components such as excipient, diluent, carrier, preservative, adjuvant or other immune enhancer, or combinations thereof, as would be readily understood by those in the art.

The present invention utilizes a novel and inventive approach to design a nano constructs which have the potential to display multiple copies of selected peptide(s) in homogenous oligomeric conformation as protein subunit vaccine with high yield in prokaryotic expression system. However, the technology of the present invention is amenable in cloning the polypeptide sequence in wide variety of prokaryotic host cell and cloning vehicles. For example, useful cloning vehicles may include bacterial plasmids, and wider host range plasmids such as pBR 322, the various pUC, pGEM and pBluescript plasmids, bacteriophages, e.g. lambda-gt-Wes, Charon 28 and the Ml 3 derived phages and vectors derived from combinations of plasmids and phage or virus DNA, such as SV40, adenovirus or polyoma virus DNA. Useful hosts may include bacterial hosts, yeasts and other fungi, plant or animal hosts, such as Chinese Hamster Ovary (CHO) cells, melanoma cells, dendritic cells, monkey cells and other hosts.

In the present invention the design of the vaccine system may include polypeptide that is flanked at C terminal with His-tag for simplified purification. In some embodiments, the present design could add other tags that eases in stabilization, secretion and purification i.e., ubiquitin tag, NusA tag, chitin binding domain, green fluorescent protein (GFP), hemagglutinin influenza virus (HAG), glutathione-S transferase (GST), streptococcal protein G, staphylococcal protein A, T7genel0, avidin/streptavidin/Strep-tag, trp E, chloramphenicol acetyltransferase, lacZ (b-Galactosidase), His-patch thioredoxin, thioredoxin, FLAG™ peptide (Sigma-Aldrich), S-tag, and T7-tag.

The present invention discloses design and development of tagless polypeptide for commercial use and thereof.

In an embodiment, the present invention provides the design and development of a vaccine specific to immunogenic and highly conserved epitopes in the form of short peptides from SARS-COV-2 spike(S) protein, which can be stitched together and stapled to a self-assembling nano cage for expression and production of higher order nanoparticle based soluble protein. In one aspect, the present invention provides an engineered or non-naturally occurring recombinant multiepitope nano protein which can be used as a vaccine candidate through intramuscular or intradermal route at certain dose to elicit desired humoral responses and provide protection. The highly conserved immunodominant domains or epitopes or peptides generally remains unchanged in the virus and a vaccine made from such conversed domains of any virus system are hypothesized to provide protection against virus-based diseases and disorders including any newly emerging SARS-COV-2 strains.

A design of a vaccine system including only the neutralizing epitopes is envisaged to facilitate in eliminating non-neutralizing antibody mediated cellular cytotoxicity.

The present application discloses that second generation SARS-COV-2 or beta corona viruses spike domain multiepitope sub unit based nano proteins may be utilized as a SARS-COV-2 or beta corona viruses vaccine candidate either alone or with adjuvants that elicits virus specific neutralizing antibodies. The present nano protein could also be used as an immunogenic antigen or as antigens for the identification of antibodies from SARS-COV-2 or beta coronaviruses infected individuals or vaccinated subjects from population, or ligand libraries for identification of small molecules, or as research reagent.

In an embodiment of the present invention there is provided a method of designing a multiepitope self-assembled nanoparticle vaccine.

The method of designing a multiepitope self-assembled nanoparticle vaccine starts with the selection of immunogenic peptides of different strains of a virus, which is further screened on the basis of their immunogenicity; and peptides showing high immunogenicity are selected.

In an embodiment, the present invention has been illustrated by utilizing the different immunogenic peptides of SARS-COV-2 virus, selected from SARS-CoV-2 receptor binding domain (RBD) peptides and heptad repeat (2) region from spike protein of SARS-COV-2 virus.

The present method can be utilized for for designing of multiepitope self-assembled nanoparticle vaccine against virus strains selected from SARS-COV-2 variants of concerns (VoC), MERS, Monkey pox virus and Dengue virus.

In another embodiment the present invention involves design and development of an engineered vaccine (DS2) against SARS-COV-2 VoC by stapling 20 mer RBD peptides from SARS-COV-2 Delta variant in the N terminal of nano cage NSP10 and conserved 20 mer peptides from the fusion peptide and 20-mer C terminal domain from SARS-COV-2 ancestral Wuhan-1 strain by linkers to the C terminal domain of NSP-10. A second immunogen can be designed by stapling the whole RBD domain of Alpha variant and Omicron variant in the N and C terminal domain of the NSP10 respectively.

Panbetacorona immunogens are designed by identifying the suitable epitopes and conducting extensive bioinformatic analysis. The epitopes and domains from SARS-COV-1, CoV-2 and MERS are stapled in different combinations for development of pan betacorona vaccine.

Similar approach was taken for the designing of vaccines against emerging Monkey pox virus and Dengue virus.

Monkey pox viruses are large, enveloped viruses. Their genome consists of a linear, double-stranded DNA (dsDNA) of ˜200 kilobase pairs and contains ˜200 genes. It is important to understand the virus biology for designing and development of novel vaccine candidates. The pox viruses produce two infectious particles: mature virions (MVs) which mainly remains intra cellular (IMV) and extracellular virions (EEVs). Both IMVs and EEVs bind to a variety of host cells using different attachment mechanisms. Approximately, 11+ proteins from IMV and EEV are collectively referred to as the entry fusion complex (EFC), which tightly associates with the viral membrane. Hence, there are more than one antigen which can be considered for the vaccine design. The initial association of IMV with the cell occurs through glycoso aminoglycan binding of the A27, D8, and H3 proteins, which are the surface antigens and suitable for vaccine designing. The association of EV with the cell surface may be mediated in part through a lectin-binding site of the A34 protein. Multiple plasmids encoding L1, A27, B5, and A33 have shown to protect mice against intraperitoneal challenge with vaccinia virus better than immunization with any individual plasmid. Hence, for development of protein subunit-based vaccine it is necessary to include more than one antigen for the development of suitable vaccine. In the present invention, the inventors have designed a monkey pox vaccine by stapling antigens B6R and A29L attached to the N terminal domain of the NSP10 and the antigens M1R and A35R are stapled to the C terminal domain of the NSP10 by using linkers.

A similar approach has been taken for designing and development of Dengue virus vaccine by using NSP10 nano cage system. Here, linear immunogenic peptides are selected from the envelope protein (E) of DENV virus surface. Three short peptides are stitched at the N-terminus of NSP10 using short linkers and four peptides were stitched at the C-terminus.

The immunogenic peptides or epitopes or domains of the virus strains are selected on the basis of their ability to induce cellular and humoral immunity and eliciting neutralizing antibody responses.

The immunogenic peptides are selected from B cell epitopes selected from nucleoprotein (NP), Matrix 1 protein (Ml), Matrix 2 protein (Ml), non-structural protein (NS), T cell epitopes selected from enveloped viral proteins or fragments, spike or envelop protein, nucleoprotein (NP), Matrix 1 protein (Ml), Matrix 2protein (M2), non-structural protein (NSP); and virions. selected from remains intra cellular (IMV) and extracellular virions (EEVs).

Further, the selected immunogenic peptides are stitched with each other by using linkers to form multipeptide or multi epitopes.

The linkers for the designing of multiepitope self-assembled nanoparticle vaccine are selected from Gly-Gly-Ser-Gly and Gly-Ser-Ser-Gly. The linkers could be gly rich linkers (Gly-Gly-Gly-Gly-Ser-Leu-Val-Pro-Gly-Ser-Gly-Gly-Gly-Gly-Ser), alanine-rich linker (Ala-Ala-Gly-Ala-Ala-Tyr-Ala-Ala), five-amino acid Gly linker (Gly-Gly-Gly-Gly-Gly) linker, other types of flexible linkers, including KESGSVSSEQLAQFRSLD and EGKSSGSGSESKST.

The present method for designing of multivalent self-assembled nanoparticle vaccine further comprises nanocage to hold the multipeptides together.

The method is provided comprising stapling of the multipeptides or multiepitopes to suitable nanocage by a linker to form a multipeptide.

The nanocage for the method is selected from non-structural protein 10 (nsp10), Ferritin, lumazine synthase (LS)

The lumazine synthase (LS) could also be used as a nano cage, which is isolated from hyperthermophile Aquifex aeolicus (AaLS). The LS forms a large nanocage consists of 60 identical subunits forming an icosahedral capsid architecture (T=1 state) with about 15.4 nm exterior and about 9 nm interior diameters, respectively.

In an embodiment of the present invention there is provided a multivalent self-assembled nanoparticle vaccine comprising
i. a multi-peptide or multi epitopes or multi domains consisting of or one or more immunogenic peptides or epitopes of domains from same virus or different viruses from the same family stitched by one or more linker; and
ii. a nanocage, wherein the multi-peptides or multiepitopes or domains are stapled with the nanocage.

The production of a vaccine largely depends on the expression of the viral peptides or epitopes or domains into a host cell. For the purpose, the multipeptide is either further flanked at the C-terminal followed by tagging or in some cases for regulatory purposes designed and purified without any tag.

The tag is selected from His-tag, ubiquitin tag, NusA tag, chitin binding domain, green fluorescent protein (GFP), hemagglutinin influenza virus (HAG), glutafhione-Stransferase (GST), streptococcal protein G, staphylococcal protein A, T7gene, avidin/streptavidin/Strep-tag, trpE, chloramphenicol acetyltransferase, lacZ (b-Galactosidase), His-patch thioredoxin, thioredoxin, FLAG™ peptide (Sigma-Aldrich), S-tag, and T7-tag.

In an embodiment of the present invention therefore there is provided tagged multi-peptide or multi epitopes nano immunogens and tagless nano immunogens for their expression in host cell.

The codons of the multipeptides or multiepitopes or domains are further optimized to obtain the encoding polynucleotides and similarly expression vectors are developed encoding polynucleotide of the multipeptides or multiepitopes of domains for its expression in a host cell to obtain a multivalent self-assembled nanoparticle vaccine.

The expression vector is selected from a plasmid such as pBR 322, pUC, pGEM, pBluescript, bacteriophages such as lambda-gt-Wes, Charon 28 and the Ml 3 derived phages and vectors derived from combinations of plasmids and phage or virus DNA, such as SV40, adenovirus or polyoma virus DNA.bacterial hosts, yeasts and other fungi, plant or animal hosts, such as Chinese Hamster Ovary (CHO) cells, melanoma cells, dendritic cells, monkey cells and other hosts.

Therefore, in an embodiment of the present invention there is provided a method of designing a multivalent self-assembled nanoparticle vaccine comprising the steps of:
i. identifying an antigenic peptide or peptide selected from one or more immunogenic peptides, epitopes, domains from the conserved region of a virus or different strains of a virus of the same family;
ii. screening one or more antigenic peptide, immunogenic peptides, epitopes or domains identified in step (i) and selection of at least one immunogenic peptide or epitope or domain or antigenic peptide showing high immunogenicity;
iii. stapling said one or more antigenic peptide, immunogenic peptide, epitope or domain screened in step (ii) by one or more linker;
iv. stitching said stapled one or more antigenic peptide, immunogenic peptide, epitope or domain of step (iii) to a nano-cage by one or more linker to obtain a multipeptide;
v. optimizing codons for expressing said stitched multipeptide to obtain an encoding polynucleotide;
vi. developing an expression vector comprising said encoding polynucleotide;
vii. expressing said vector in a cell to obtain a multivalent self-assembled nanoparticle vaccine.

The design of the multivalent Self-assembled Nanoparticle vaccine platform (MSN-vaccine platform) of the present invention is illustrated by utilizing SARS-CoV-2 immunogenic peptides.

In an embodiment of the present invention there is provided an engineered SARS-CoV-2 nano protein immunogen which is expressed from a polynucleotide comprising a coding region encoding a polypeptide, wherein said polypeptide comprises at least two or more SARS-COV-2 receptor binding domain (RBD) peptides or two or more peptides from heptad repeat (2) region from spike protein linked by linkers.

The designing and development are further extended to include epitopes from different virus strains or variants of SARS-COV-2 such as inclusion of epitopes from Delta, Alpha and Omicron.

In an embodiment of the present invention there is provided designing and development of Panbetacorona immunogens by incorporating the epitopes or domains from three or four viruses from the Coronavirus family i.e. SARS-COV-1, SARS-COV-2 ancestral Wu-1 or variants of concerns, and MERS.

In an embodiment of the present invention there is also provided designing and development of a vaccine against Monkey pox virus by using multiple envelop antigens and stapling to NSP-10 nano cage.

In an embodiment of the present invention there is further provided designing and development of a multivalent Dengue vaccine by stapling the conserved B cell epitopes from DENV-2 serotype to NSP-10.

### Design of Multivalent Self-Assembled Nanoparticle Vaccine Platform (MSN-Vaccine Platform)

### A. Selection of the Candidate Epitopes for Self Assembly (SARS-COV-2)

### The Protein Tested for This Selection—

SARS-COV-2 is an enveloped, RNA virus that belongs to the family Coronaviridae. The SARS-COV-2 S protein is a type I transmembrane glycoprotein which remains in a metastable conformation and three homo-dimeric complexes assemble to form trimers that are exposed on the virion surface. The S glycoprotein initiates the infection by attachment to hACE2 receptor present on the host cell surface, thus stimulating conformational changes for the fusion of the viral-host cell membrane. Thus, the SARS COV-2 S protein is the major target for eliciting neutralizing antibodies and to confer protection. The majority of present available SARS-COV-2 vaccine candidates are mainly based Spike based vaccine candidates. The S protein is a 180 kDa glycoprotein and contains three major domains; the ectodomain (S1 and S2 domains), transmembrane domain, and intracellular cytoplasmic domain. The S1 domain consists of N-terminal domain (NTD) and receptor-binding domain (RBD) that binds to the host cell receptor hACE2, and initiates attachment. The C-terminal domain (CTD); i.e., the S2 domain which contains the highly conserved fusion peptide and heptad repeat (HR) domains, mediates fusion process and facilitates virus entry into the host cell. Present invention selects the spike protein to establish the multi epitope based nano vaccine platform.

### Bioinformatics Study

Immune-bioinformatic analysis by using B cell linear epitope prediction, Bepipred Linear Epitope Prediction 2.0 for antigenicity prediction and solubility is done for identification of epitopes (immunogenic peptides) from the sequence of SARS-CoV-2 Wuhan-1 spike protein. Computation methods is also used on machine learning to select promising and important peptides (B cell and T cell epitopes) or class I HLAs to be presented on the host's cell surface and the immunogenicity of these peptides. The peptides that were selected from the SARS-COV-2 primary amino acid sequence were missing in the available structures in the protein data bank for the SARS-COV-2 spike protein. This suggests that the regions from where the peptides selected are random and have a high b factor. To build-up the missing parts, SWISS-MODEL and PDB:6VYB as a template have been used. The structure of SARS-COV-2 ectodomain in PDB:6VYB is in an open state, with one RBD open while the two are in the closed conformation ((Walls A C et.al. 2020).). This helped us to view the state of RBD-pep2 on the SARS-COV-2 structure both in the context of open and closed conformation. The location and conformation of both, S1-pep1 and RBD-pep2 were highly exposed and easily accessible on the SARS-COV-2 ectodomain, suggesting that antibodies for these regions have easy accessibility on the S protein. The RBD-pep2 is part of the RBM which in general, is very flexible and adopts a structure when binds to the ACE2 receptor Antibodies targeting the RBM region are neutralizing, and it was hypothesized that the use of RBD-pep2 as a peptide vaccine candidate might elicit neutralizing antibodies. To build the missing region of S2-pep3 on the S protein, int-FOLD server (Mcguffin L J et.al, Nucleic Acids Res. (2019) 47:W408-13) was used to model the missing C-terminal domain of S2 region. The best fit modeled S2-pep3 region shows that it forms a helix and projects upward from the proximal region of the membrane. The conserved hydrophobic region that immediately precedes the trans-membrane region is also crucial for S-protein trimerization and stability. Antibodies binding to this region may destabilize the trimer formation or post-fusion conformation (Schroth-Diez B et.al, Biosci Rep. (2000) 20:571-95). A similar phenomenon is hypothesized in HIV envelope protein, where the region closer to the membrane where the region closer to the membrane induces antibodies that block the conversion from pre-fusion to post-fusion conformation during the process of cell entry (Williams K L et.al, PLOS Pathog. (2019) 15:e1007572).

### Scoring of Antigenicity

i. Predicted epitopes were subjected to Vaxijen 2.0 for antigenicity test (Irini A. Doytchinova, and Darren, R Flower et.al, 2008 Open Vaccine J. 2008; 1(1):22-6.)
ii. Structural and molecular docking studies
iii. Immunogenicity screening (peptide showing high binding antibodies)
iv. Ability of the peptide to stimulate and show T cell responses

### Experimental of Three Peptides:

### I. Immunization of Peptides in Mice Induces High-Titer Antibody Responses

To determine the immunogenicity of the peptides, BALB/c mice, 6-8-weeks-old were immunized. The whole IgG responses of the immunized sera from different experimental groups and one control group were measured against the RBD-peptide and HR2 peptide by ELISA. The immunized sera from all the groups were able to elicited whole IgG titers. The control group with PBS did not show reactivity to any of the peptides. The peptide-specific IgG subclass switching in sera from the second boost immunization to determine Th1/Th2 polarization were also investigated. IgG1 subclass was found to be dominated in the sera from the immunized mice. whereas, no titer was detected against IgG2c subclass (Vishwakarma P. et.al, Front Immunol. 2021 Mar. 26; 12:613045, Yadav N et.al Microbes Infect. 2021 May-June; 23(4-5):104843)

### II. Peptide Prime-Boost Immunization Efficiently Mounted Antigen-Specific CD8+T Cell Responses

The T-cell responses to the immunized peptide in the mice were also characterized. For this purpose, spleens were isolated from each of the immunized groups and stimulated in vitro in the presence of PMA+Ionomycin as control and respective peptide antigen and compared with the mock immunized control group. The characterization of various T-cell populations was carried out based on the presence of CD4, CD8 surface markers, and cytokines. In peptide stimulated groups, both the T helper as well as T cytotoxic cells showed ˜6-fold upregulation as compared to the control group for RBD peptide. However, no significant changes were observed for the HR2 peptide immunization.

### III. Functional and Biological Characterization of SARS-COV-2 Peptide Immunized Sera

To determine the soluble spike protein (in pre-fusion stabilized conformation) and soluble RBD-specific antibody titers of the peptides immunized sera from the second boost. The highest total IgG titers against the soluble spike protein were seen against both the RBD peptide and the HR2 peptide immunized mice pooled sera. These data corroborate with the earlier results that HR2 peptide are highly immunogenic in BALB/c mice. To further confirm whether the induced antibodies recognize the conformational spike protein, Ni-NTA HisSorb ELISA plates were used to coat spike protein, and ELISA was repeated with RBD-peptide and HR2 peptide respectively. The sera from both the peptides showed binding to conformationally stable spike protein and interestingly, the sera showed higher binding titers to soluble spike protein in Ni-NTA coated plates as compared to the binding titer to ELISA. The IgGs were also purified from the immunized sera mice to assess their binding with a soluble spike in pre-fusion conformation in real-time using bio-layer interferometry (BLI). All the sera were found to bind with the soluble spike protein; this further confirms that the immunized sera have antibodies that are specific to the spike protein of SARSCOV-2 and bind to the soluble, pre-fusion, native-like, trimeric spike protein. The ability of the immunized pooled sera to bind selectively to soluble spike and RBD protein by Western blot was analysed. The sera from the RBD peptide immunized group efficiently detected the soluble spike protein at 1:100 dilution as 175 KDa band and Soluble RBD at 29 KDa. Similarly sera from the S2 peptide immunized group detected soluble spike protein at 1:1000 dilution as 175 KDa band.

### IV. Neutralizing Responses of the Anti-Peptide Sera to Ancestral Wu-1 SARS-CoV-2 Virus

The ability of immunized sera of peptides to neutralize the SARS-COV-2 was evaluated by using classical virus plaque based neutralization assay in Vero E6 cells. The sera from the peptide of immunized groups after the second boost were incubated with 50 PFU of SARS-COV-2 (USA-WA1/2020isolate) at 1:10 and 1:20 dilutions, respectively and 48 h p.i. The number of plaques formed was visualized by crystal violet staining. The sera from all groups immunized with peptide showed to neutralize the SARS-COV-2 virus as compared to the immunized sera of the control group. The ability of the RBD-peptide immunized sera to block ACE2-RBD interaction by using surrogate neutralization assay was tested. At 1:10 dilution, the sera from the RBD-peptide immunized mice from the second boost showed about 17% inhibition in binding of RBD to ACE2 in competitive ELISA. The result was in agreement with plaque reduction neutralization test (PRNT) indicating that the 20-mer RBD-peptide harbors the neutralizing epitope. Although, the neutralizing titers were low but this is attributed to the small size or low dose of the peptides or both, and the adjuvant used. Nevertheless, the 20-mer peptides were able to induce neutralizing responses albeit in weak concentration.

### Selection of the Immunogenic Peptide or Epitopes:

The envelope spike protein of SARS-COV-2 is the major target to induce protective immune response in the host. This is evident and well documented in the from the literature that protective neutralizing antibodies targeting the spike protein are present in the samples from convalescent patients. These neutralizing antibodies are elicited against different domains of the spike protein. The complete chain of spike protein of SARS-COV-2 (Wuhan-Hu-1 strain, GenBank accession ID: MN908947.3) were analyzed for the identification of immunogenic peptide from three distinct different regions, the n-terminal domain (NTD), the receptor binding domain (RBD) and the S2 domain. The protein chain was analyzed for immunogenic peptides using immune-informatic approach for B-cell linear epitope prediction and peptides with favorable values for the predicted antigenicity were selected. Two peptides one from the RBD region and one from the S2 domain were selected. The peptide selected from the RBD is a part of the receptor-binding motif (RBM). Not only these peptides showed high antigenic value but the corresponding peptide from the SARS-COV is also known to bind to neutralizing antibodies, such as F26G18 and F26G8 (24). The second peptide selected from the C-terminal region of S2 domain, the HR2, is reported previously as effective viral inhibitor by Tripet et al J Struct Biol. (2006) 155:176-94). The antigens are scored as described above of their antigenicity and structural modelling is conducted. The immunogenicity results from two studies (Vishwakarma P et.al, Front Immunol. 2021 Mar. 26; 12:613045and Yadav. N et.al, 2021 Microbes Infect. 2021 May-June; 23(4-5):104843) are further evaluated and accordingly two peptides are chosen for further development of nano vaccine (peptide from RBD and peptide from HR2 domain).

### B. Selection of Suitable Nano Cage

### NSP10 as Nano Cage:

The non-structural protein 10 (nsp10) has potential interest as a nanoparticle particularly for SARS-COV-2 based vaccine design platform due to its presence within the coronavirus genus, including the SARS-COV-2 virus. NSP10 is a 17 kDa protein which can display monomeric peptide antigens, each set in the N-or C-terminal and self-assembled into a spherical dodecahedral nanoparticle with twelve faces. Interestingly both the N-and C terminals in Nsp10 nanocage are surface-exposed and are located on separate threefold axes. This conformation allows for the attachment of antigen(s) at both terminals. NSP-10 has been selected as it is highly conserved in beta coronavirus and its ability to induce T cell immune responses.

### Ferritin:

Ferritin is a popular non-viral platform used previously for designing nanoparticle based vaccine and therapeutics. Ferritin is involved in intracellular iron storage that is found in nearly all organisms and consists of 24 monomers each with a molecular weight of 18 kDa. The complex consists of 8 trimers with octahedral symmetry and resembles a rhombic dodecahedron. The N-termini is in close proximity of to the threefold axis allows for easy attachment of trimeric antigens. However, the C-terminus is buried and unavailable for antigen presentation. Helicobacter pylori ferritin has been commonly used in vaccine design due to its sequence divergence from human ferritin.

### Lumazine Synthase Protein

Luminase synthase has been isolated from hyperthermophile Aquifex aeolicus (AaLS), and consists of 60 identical subunits forming an icosahedral capsid architecture (T=1 state) with a 15.4 nm exterior and a 9 nm interior diameters, respectively. This enzyme catalyzes the penultimate step in the biosynthesis of riboflavin, also generally known as vitamin B2. By using recombinant technology, lumazine synthase subunit could be fused with antigenic epitopes or domains and the immunogenic protein could be produced by in a suitable prokaryotic or eukaryotic cell culture system.

### C. Construction of the Plasmid

i. Choice of Linker:

To connect two domains of protein or two or more peptide or epitopes flexible linker is preferred to provide certain degree of free movement of the joined domains or peptides or interaction. For this purpose, small polar amino acid such as Glycine and, non-polar amino acid such as Serine is preferred. The small size of these amino acids provides flexibility, and allows for mobility of the connecting functional domains. The incorporation of Ser helps in maintaining the stability of the linker in aqueous solutions by forming hydrogen bonds with the water molecules, and reduces the unfavorable interaction between the linker and the protein moieties. Bioinformatic analysis with different lengths of linkers, Gly-Gly-Ser-Gly and Gly-Ser-Ser-Gly were selected as linkers of approximate length of 2.2-2.6 Å. The length is optimal for free rotation of the stapled peptides and same time marinating sufficient structural conformation and improved solubility of the designed protein constructs.

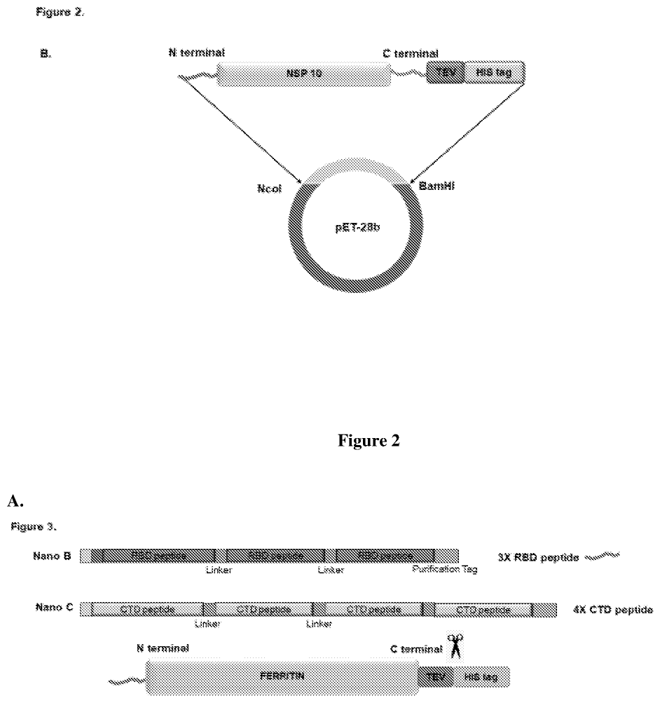

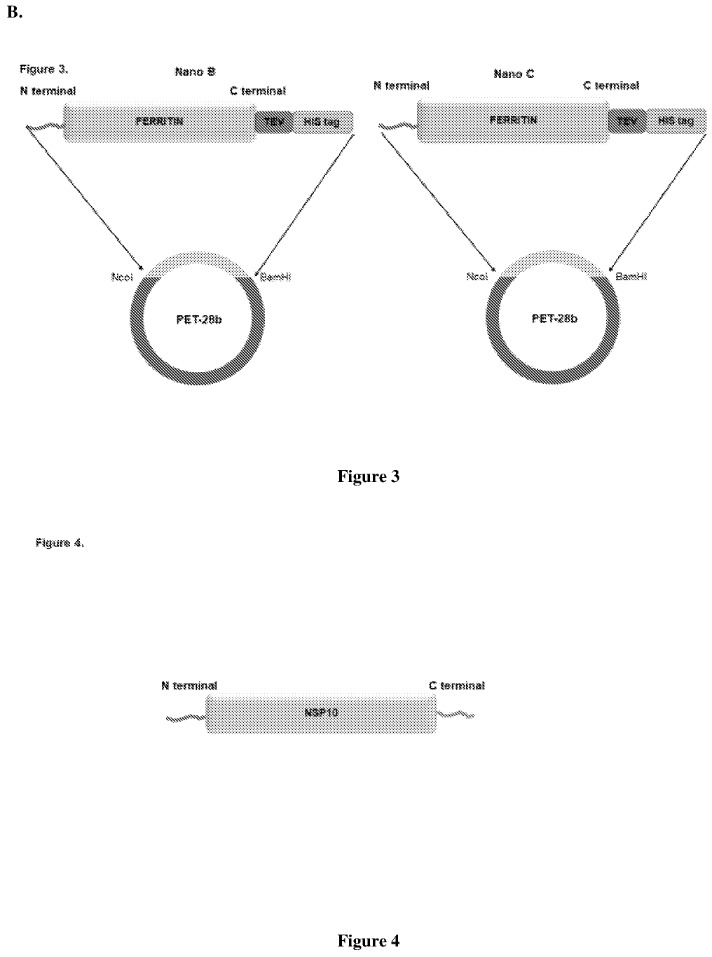

ii. Construction of the Plasmid With Immunogenic Epitope Sequence, Linker and Subsequently With the Nano Cage
The developed nanovaccine henceforth called as Nano A is comprised of maximum identity with the amino acid sequence encoded by SEQ ID No. 1, SEQ ID No. 2, SEQ ID No. 3 and SEQ ID No. 4 as described later in the embodiments. Two repeats of the SEQ ID No.1 are linked together by linker SEQ ID No.3 and the polypeptide is linked to SEQ 1D No. 4 in the N terminal and two repeats of the SEQ ID No.2 are linked together by linker SEQ ID No.3, the stapled polypeptide has been linked to the SEQ 1D No. 4 in the C terminal to form polypeptide Nano A designated as SEQ ID No. 5 which is further flanked by Tobacco Etch Virus (TEV) protease and 6×-Histidine tag at C terminal end of the polypeptide.
In Silico codon adaptation and cloning (For high expression of the vaccine in the suitable host that will be cost effective and scalable production. The codon optimization of Nano A polypeptide was conducted in Java Codon Adaptation Tool (JCat) to test the increase the translational expression in E. coli K12 system. The codon adaptation index (CAI) value and GC content of the adapted sequence were further analysed and assessed it within a particular threshold. By using SnapGene 4.2 tool. Thereafter, the obtained codon optimized polypeptide was cloned into the pET28a vector for expression in Escherichia coli as described in FIG. 2.
The polypeptide designated as Nano B has been designed by linking three repeats of the SEQ ID No.1 by linker SEQ ID No.3 and the polypeptide is linked to SEQ 1D No. 6 in the N terminal to form the Nano B polypeptide. The C terminal end consists of Tobacco Etch Virus (TEV) protease and 6×-Histidine. The whole polypeptide was codon optimized as describe above and was cloned into the pET28a vector for expression in Escherichia coli as described in FIG. 3A.
The polypeptide designated as Nano C has been designed by linking four repeats of the SEQ ID No.2 by linker SEQ ID No.3 and the polypeptide is linked to SEQ 1D No. 6 in the C terminal to form the Nano C polypeptide. The C terminal end consists of Tobacco Etch Virus (TEV) protease and 6×-Histidine. The whole polypeptide was codon optimized as describe above and was cloned into the pET28a vector for expression in Escherichia coli as described in FIG. 3B.

### D. Expression and Purification of the Expressed Protein

The cloned plasmids were propagated in the DH5-alpha strain of E. coli. For protein expression the plasmid with the sequence of interest were transformed in Rosetta (DE3) strain of E. coli. After transformation, the Rosetta (DE3) strain of E. coli carrying the plasmid was grown in the super broth medium and overexpression of protein was induced at OD of 0.6 using 0.5-1.0 mM and further cultured at 18° C. to 37° C. for 4 hrs. Fractions of induced culture were collected at different time intervals, the samples were separated on a 12% Bis-Tris SDS polyacrylamide gel. The Nano A construct showed overexpression as seen in Annexure-I. The overexpression of protein of interest is shown in the red square. The eluted fractions were separated on a 12% Bis-Tris SDS polyacrylamide gel. On the SDS-PAGE the protein Nano A showed clean band in soluble fraction confirming the purification. The approximate molecular weight of Nano A C is around ˜29 kDa (as per the sequence Nano A molecular weight is around 28083.40 kDa and and Nano C molecular weight is around 30726.31 kDa. The nano B construct was not expressed in soluble fraction. As used herein, the term “purified” means that the polynucleotide, polypeptide, protein, variant, or derivative thereof is substantially free of other biological material with which it is naturally associated, or free from other biological materials derived, e.g., from a recombinant host cell that has been genetically engineered to produce proteins of the invention.

The soluble Nano immunogen with His tag (Nano A in NSP10 nano cage, Nano B RBD—in Ferritin nano cage, Nano C HR2—in ferritin nano cage) are expressed, purified and characterized. The Nano A protein is found in the soluble fraction and the yield was around ˜70 mg/liter. The Nano B protein was found in inclusion bodies and Nano C protein was found in soluble fraction and the yield was around ˜10 mg/liter. The nano proteins A and C were further characterized and were found to bind to commercially available anti-His antibody and polyclonal anti-RBD antibody raised in-house in mouse by administration of whole soluble RBD protein along with Addavax™ adjuvant. Additionally, it was found that the expressed proteins are forming oligomers in native page thus suggesting formation of self-assembled nano particle.

### Difficulties of Secretion in Soluble Form

Expressing viral protein in prokaryotic system is challenging due to presence of disulphide bond, glycosylation and differential use of codon. To achieve high expression the whole polypeptide chain was codon optimized and linear epitopes were selected to express in prokaryotic expression system in conjugation with nano cages for displaying epitopes on the surface of nano cages. The design enabled high level of expression in prokaryotic expression system e.g E. Coli. With an estimated yield of 70-100 mg/lt of super broth culture in standard shaking flask. To purify the protein of interest to homogeneity, ammonium sulphate precipitation, followed by ion exchange column and gel filtration chromatography.

Given the high yield of the protein in the bacterial expression system and ease of purification, the system is easy to scalable for high volume culture using fermenters to express and purify large amount of Nano A protein.

### E. Characterisation of Protein

The Nano construct contain NSP-10 (Nano A) or Ferritin (Nano C) which self assembles in too well ordered nano-particles. To confirm their tertiary structure and oligomeric status 4-12% gradient Blue Native PAGE was run. The Nano A protein form an oligomer with expected molecular weight of approximately ˜140 kDa suggesting pentameric structure and Nano C with a molecular weight of approximately ˜800 kDa suggesting formation of higher order oligomers.

The Nano A protein purified by Ni-NTA column was concentrated and loaded on a Superdex 16/60 size exclusion chromatography column equilibrated with PBS. A single peak at elution volume of 15 ml eluted for the SEC column corresponded to a molecular weight of approximately ˜140 kDa. The identity of the eluted fraction was confirmed by SDS-Page analysis and Western blot using HRP conjugated anti-His antibody and polyclonal sera of anti-RBD. The results suggest the Nano A protein forms oligomeric self-assembled nano structure and there is minimal aggregation.

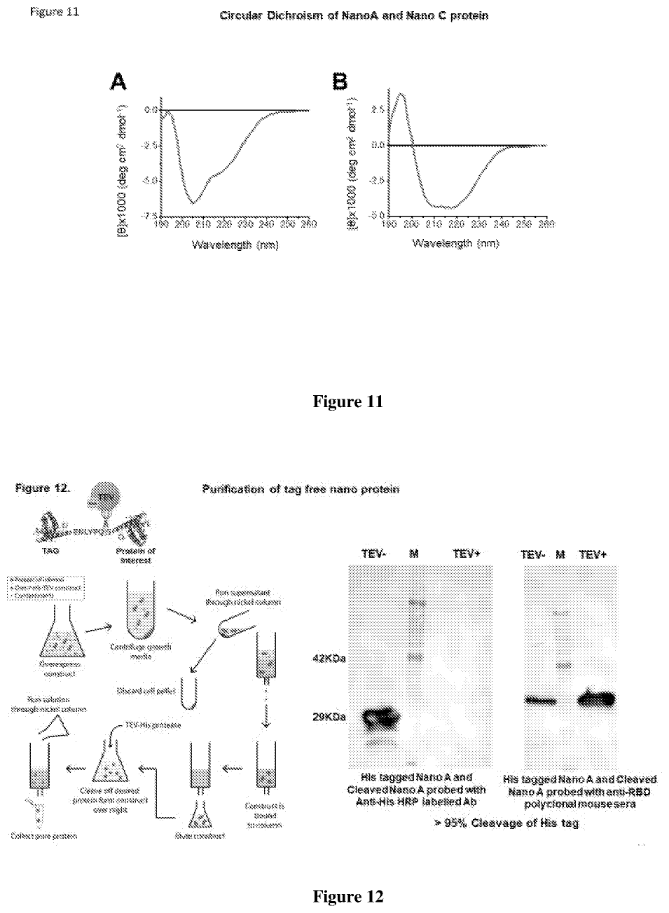

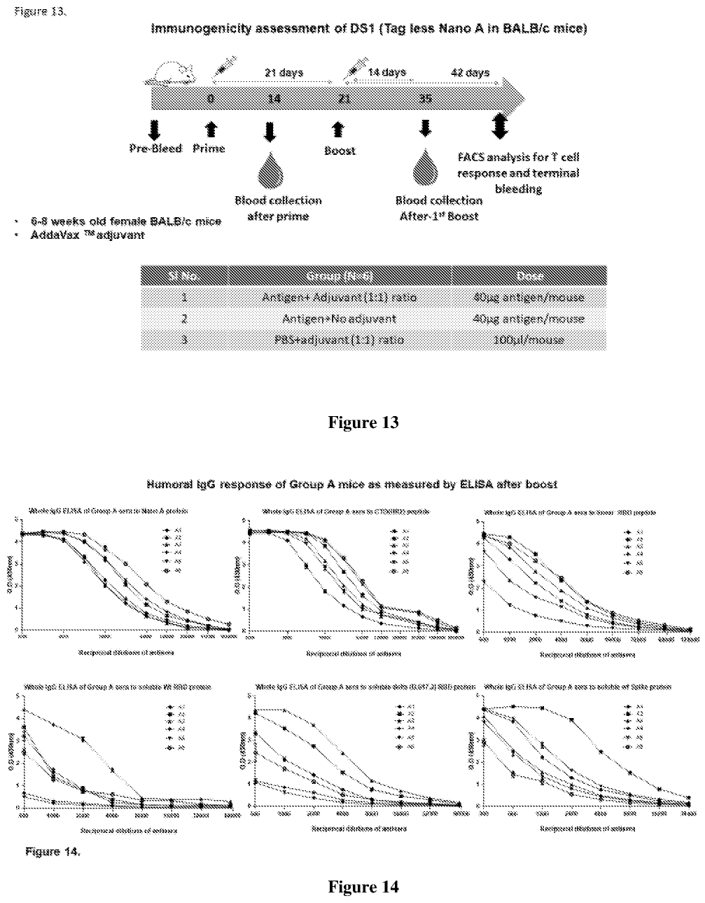

The secondary structure signature of both purified Nano A and Nano C protein was determined by CD spectra analysis. Far-UV CD spectra were acquired on a Jasco-815 spectropolarimeter. The concentration of the protein used is 5 μM. Cuvette of path length of 0.2 cm was used and spectra were collected from 260 to 190 nm at a rate of 100 nm/min and data pitch of 1 nm for each protein, with averaging of 10 scans for noise reduction. Contributions of the buffer to the spectra were electronically subtracted and for each spectrum, mean residual ellipticity (MRE) was calculated and plotted. The secondary structure signature of Nano A protein is showing peak minima at about 208 nm suggesting (FIG. 11A) suggesting it is predominantly β-sheet rich protein and small amount of α-helix as indicated by peak minima contribution at 222 nm. The Nano C protein is predominantly α-helix with peak minima at 222 nm (FIG. 11B).

### G. Formulation of the Nano Protein Into a Composition

The invention further provides a composition by adding adjuvants to the Nano protein and a method of administration to a “subject” to induce immune responses. The term “subject” is meant any subject, particularly a mammalian subject, for whom diagnosis, prognosis, immunization, or therapy is desired. Mammalian subjects include, but are not limited to, humans, domestic animals, farm animals, pet animals such as dogs, cats, guinea pigs, rabbits, rats, mice, horses, cattle, cows; primates such as apes, monkeys, orangutans, and chimpanzees; food animals such as cows, pigs, and sheep; rodents such as mice, rats, hamsters and guinea pigs; and so on. In certain embodiments, the animal is a human subject. The term “animal” is intended to encompass a singular “animal” as well as plural “animals” and comprises mammals and birds. The term animal also encompasses model animals, e.g., disease model animals. In particular, the mammal can be a human subject, a food animal or a companion animal.

The term “adjuvant” refers to any material having the ability to (1) alter or increase the immune response to a particular antigen or (2) increase or aid an effect of a pharmacological agent. As used herein, any compound which may increase the expression, antigenicity or immunogenicity of a multi epitope nano protein of the invention is a potential adjuvant. In some embodiments, the term adjuvant refers to a TLR agonists and other adjuvants, wherein the TLR adjuvant includes compounds that stimulate the TLR receptors {e.g., TLR1-TLR13), resulting in an increased immune system response to the vaccine composition of the present invention. TLR adjuvants include, but are not limited to, CpG and MPL. Adjuvant also refer to plant based adjuvants, squalene based emulsion adjuvants, aluminum salt-based adjuvants,

### H. Immunogenicity Assessment of Nano Vaccine Candidate

The immunogenicity assessment refers to administration of the Nano vaccine candidate in nonclinical animals in effective dose (for example here in BALB/c or C57B1/6 mice) to elicit humoral and cellular response. An “effective dose” is that amount the administration of which to an individual, either in a single dose or as part of a series, is effective for treatment or prevention. An amount is effective, for example, when its administration results in a reduced virus growth invitro (for example treating the immunized sera with pseudo or live virus that will decrease or completely inhibit the viral replication in cell lines as compared to virus control) or incidence of SARS-COV-2 virus infection relative to an untreated animal or individual, as determined two weeks after challenge with an infectious SARS-COV-2 virus as reducing virus spread. This amount varies depending upon the health and physical condition of the animal or individual to be treated, the taxonomic group of individual to be treated (e.g. human, nonhuman primate, primate, etc.), the responsive capacity of the individual's immune system, the degree of protection desired, the formulation of the vaccine, a professional assessment of the medical situation, and other relevant factors. The immunization regimen could be one prime-one boost or one prime-two boosts. The terms “prime” or “priming” or “primary” and “boost” or “boosting” as used herein to refer to the initial and subsequent immunizations, respectively, i.e., in accordance with the definitions these terms normally have in immunology. However, in certain embodiments, e.g., where the priming component and boosting component are in a single formulation, initial and subsequent immunizations may not be necessary as both the “prime” and the “boost” compositions are administered simultaneously. As used herein, an “immune response” refers to a response in the recipient to the introduction of the Nano protein or composition of the present invention, generally characterized by, but not limited to, production of antibodies and/or T cells.

A protein nano cage having self-assembly properties has been used for displaying stapled multi epitopes or peptides and expressed in prokaryotic expression system as soluble proteins to be used for low cost, large scale industrial production and expect to give protection against SARS-COV-2 variants. The designing could also be used for development of cancer vaccines using neo antigens, T cell vaccines using B and T cell epitopes, usage of bacterial or parasites antigens and other

RNA viruses. The present invention provides methods of designing, production, characterization and using inventive vaccine nano immunogens and pharmaceutical compositions thereof. B-cell epitope can be obtained from any spike or envelope proteins from enveloped virus or fragments thereof from other viral proteins, e.g., nucleoprotein (NP), Matrix 1 protein (Ml), Matrix 2 protein (Ml), non-structural protein (NS).

T-cell epitopes could be used in the present invention design that can comprise any number of amino acids and be derived from any known antigens or immunogens. In one embodiment, T-cell epitopes can be derived from any enveloped viral proteins or fragments thereof, e.g., spike or envelop protein, nucleoprotein (NP), Matrix 1 protein (Ml), Matrix 2 protein (M2), non-structural protein (NSP). In a particular embodiment, T-helper cell epitopes can contain 9 core amino acids with 3 flanking amino acids on each side for a total of 15 amino acids. Its binding to the clefts of the Major Histocompatibility Complex (MHC in mice, HLA in humans) can be calculated by the known methods. The high-scoring peptides are predicted to be ligands for those MHC of HLA molecules.

### Advantages

In addition, attempts are made to express full or part of RBD or Spike protein. In this project, the antigenic epitopes have been stitched and introduced into the nano cage to express as higher order proteins. Furthermore, the proteins are expressed in bacterial expression system and in soluble fraction which aids in large scale industrial production, thus enabling development of low-cost vaccine which will be very beneficial for low-middle-income countries. It will also increase the vaccine coverage. The usage of short conserved or less variable region enables to give protection against broad range of SARS-COV-2 variants without changing the immunogen too often.

The usage of NSP-10 and ferritin as nano cage to be used for the expression of antigen has been described by Daniel C Carter.

This platform has several advantages as follows; i) Single domain peptides with minimal length have advantages as these are easy to produce, cost-effective, less complex, and easy to remove the unwanted non-neutralizing epitopes or domains. ii) The stapled immunogenic region could be tailored to introduce B cell or T cell epitopes, conserved domains, highly antigenic regions. iii) Displaying the epitopes as multimeric nanoparticles in protein cages to develop as a soluble subunit protein vaccine is a unique vaccine platform.iv) Bacterial expression and purification system is a well-established platform and provides a cost-effective means to develop and deliver vaccines and has scope for large scale commercial production. v) This platform has broad usage and could also be utilize for other infectious diseases (emerging or pandemic potential viruses or bacterial diseases or displaying cancer antigens for onco-therapy). vi) This platform utilizes only the epitopes or short antigenic peptides, hence could be easy to handle in biosafety 1 or 2 level and does not need any sophisticated equipment or laboratory structure. vii) Multimeric antigens as nano vaccines reduce the antigen doses for immunization and thus further reduces the cost of vaccine.

### Example 1: Multivalent Self-Assembled Nanoparticle Vaccine Platform Prototype Example as SARS-COV2 Multiepitope Self-Assemble Nano Immunogens

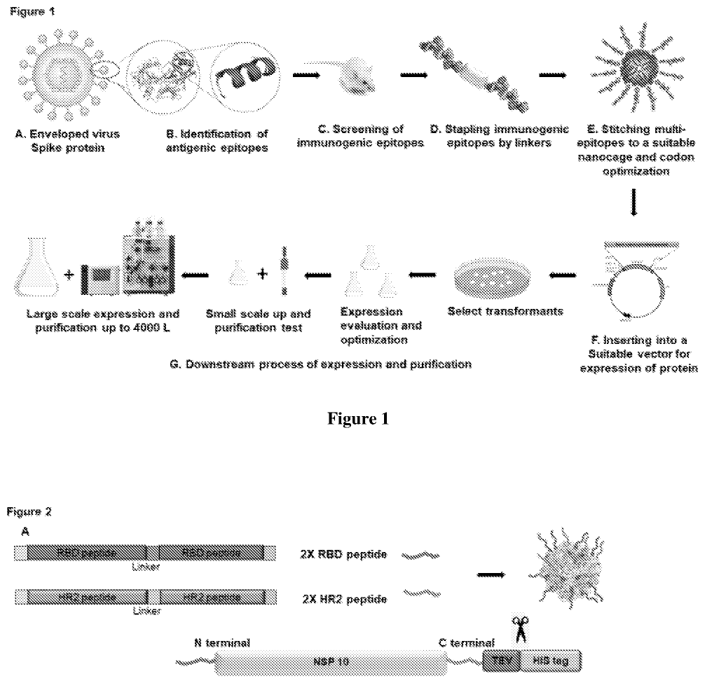

FIG. 1 depicts development of Multiepitope Self-assembled Nanoparticle vaccine platform (MSN-vaccine platform), by utilizing minimal antigenic, immunogenic or highly conserved component, or subunit, that can be utilized for the development of next generation vaccine candidates which has broad and heterotypic protection. Single domain peptides or epitopes with minimal length could be stitched together and these epitopes could be displayed as multimeric in a suitable nanocage to develop as a soluble subunit protein vaccine. The polypeptide could be expressed in prokaryotic expression and purification system which is a well-established platform and provides a cost-effective means to develop and deliver vaccines In addition, these candidates can be made via biosynthetic pathway and the subunit approach results in the development of non-infectious, chemically and physically more stable and easier and cheaper production, with highly characterize to avoid batch-to batch inconsistency compared to the recent vaccine development approach.

### Example 2: Construction of Nano A

As shown in FIG. 2, SARS-COV-2 spike protein has been stapled to two repeats of 20 mer RBD peptide and two repeats of conserved 20 mer peptide from S2 domain (conserved heptad repeat 2) by linkers. These stapled peptides incorporated into NSP-10 protein in both N terminus consisting of stapled RBD peptide and C terminus consisting of HR2 peptides. In addition, the C terminal of the polypeptide also consists of TEV protease cleavage site followed by 6×His tag for ease in purification (FIG. 2A). The complete polypeptide sequence is codon optimized and was cloned in commercially available pET28b(+) vector between Nco1 and BamH1 restriction sites (FIG. 2B. The correct cloning and sequence were confirmed by DNA sequencing.

### Example 3: Construction of Nano B and Nano C

FIG. 3 shows three repeats of 20 mer RBD peptides linked by linker and further stapled to the N-terminus of H. pylori ferritin to be presented on the threefold axis points of the H. pylori ferritin core and the C terminal consists of TEV cleavage site followed by 6×His tag (FIG. 3A, upper panel). The bacterioferritin (FR), self-assembles to form nanocages with octahedral symmetry. Ferritin is a 24-mer protein assembly to form a cage-like structure in a way similar to SARS-COV-2. It has a unique structure; hence ferritin is a promising nanoplatform for antigen presentation and immune stimulation. In FIG. 3A (lower panel) consists of a nano construct designated as Nano C in which four repeats of 20 mer HR2 peptides linked by linker and further stapled to the N-terminus of H. pylori ferritin to be presented on the threefold axis points of the H. pylori ferritin core and the C terminal consists of TEV cleavage site followed by 6×His tag. The designed polypeptides are codon optimized and was cloned in commercially available pET28b(+) vector between Nco1 and BamH1 restriction sites. The correct cloning and sequence were confirmed by DNA sequencing as illustrated in FIG. 3B upper and lower panel respectively.

### Example 4: Designing of Tagless Construct

FIG. 4 shows designing of a nano construct designated as Nano A1 which contains polypeptide sequence of Nano A however the polypeptide is without TEV cleavage site and His tag sequence at C terminal end. For industrial and regulatory purpose, the construct was designed and developed.

### Example 5: Expression of Nano A, B, C in E. coli in Pellet (Insoluble Fraction) And Supernatant (Soluble Fraction)

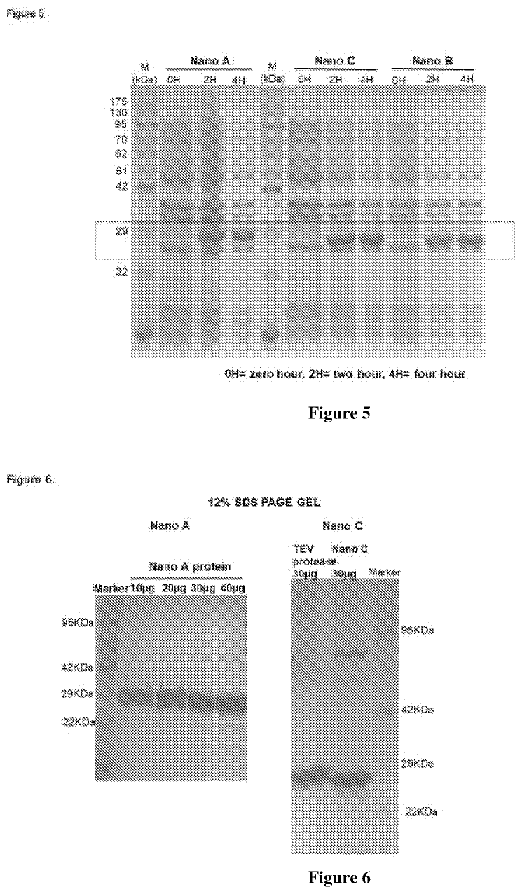

The cloned plasmids were propagated in the DH5-alpha strain of E. coli. For protein expression the plasmid with the sequence of interest were transformed in Rosetta (DE3) strain of E. coli. After transformation, the Rosetta (DE3) strain of E. coli. carrying the plasmid was grown in the super broth medium and overexpression of protein was induced at OD of 0.6 using 0.5-1.0 mM and further cultured at 18° C. for overnight or 37° C. for 4 hrs. Fractions of induced culture were collected at different time interval at OH (Uninduced), 2H and 4H and were subsequently mixed with 4× loading dye and denatured at 95° C. for 5 min. The samples were separated on a 12% Bis-Tris SDS polyacrylamide gel. All the constructs Nano A, B and C showed overexpression as seen in FIG. 5. The overexpression of protein of interest is shown in the red square.

### Example 6: Purification of Proteins Nano A and Nano C

For protein purification the pelleted cells were resuspended in PBS buffer and lysed by sonication. The lysed protein fraction was clarified by centrifugation and the clear lysate was loaded on Ni-NTA column. The column was washed with 5 column volume of PBS containing 30 mM imidazole. Bound protein from the column was eluted with 500 mM imidazole in PBS. The eluted fractions were separated on a 12% Bis-Tris SDS polyacrylamide gel. On the SDS-PAGE both the protein Nano A and Nano C showed a clean band without any contamination confirming the purification. The approximate molecular weight of Nano A and Nano C is around ˜29 kDa (as per the sequence Nano A molecular weight is around 28083.40 kDa and Nano C molecular weight is around 30726.31 kDa as shown in FIG. 6. The nano B construct was not expressed in soluble fraction.

### Example 7: Western Blot Analysis of Nano A, B, C Probed With Anti-His Commercial Antibody

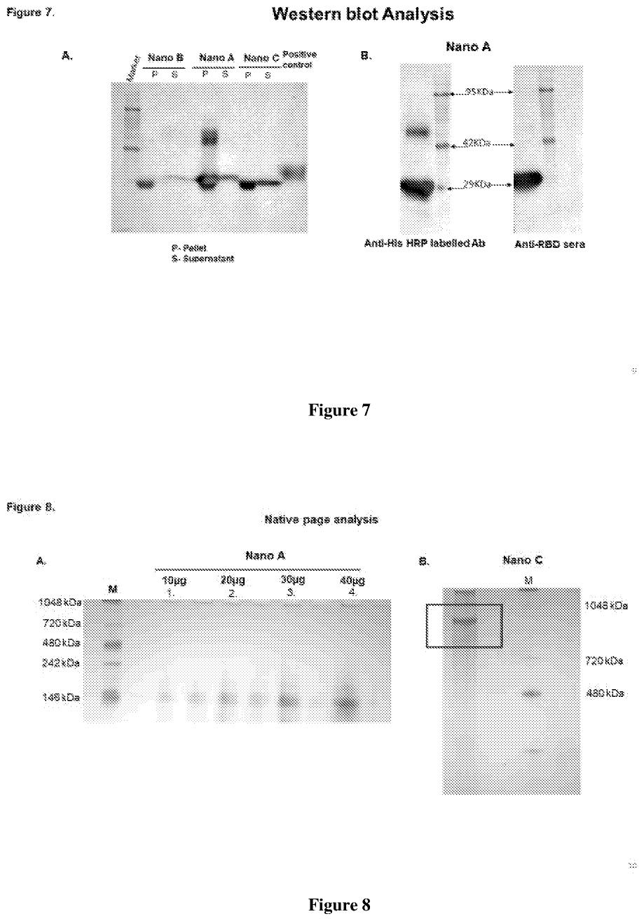

For Western blot analysis, the Nano A, B and C protein were separated in 12% SDS-PAGE and transferred to a polyvinylidene fluoride (PVDF) membrane. The membrane was blocked with 5% skimmed milk, incubated either with HRP conjugated anti-His antibody or with mice anti-RBD polyclonal sera. In case of anti-RBD sera the membrane was developed with HRP-conjugated anti-mouse secondary antibody (Jackson Immuno Research, PA, USA). Both the antibodies anti His and anti RBD polyclonal sera (mouse administered with whole soluble RBD protein) showed binding with the protein of interest in the Western blot confirming the identity of the protein. The results are presented in FIG. 7. Nano A soluble fraction is probed with anti-His antibody and anti-RBD polyclonal antibody

### Example 8: Native Page Analysis of Nano A, Nano B and Nano C Nano Proteins

The Nano construct contain NSP-10 (Nano A) or Ferritin (Nano C) which self assembles in two well-ordered nano-particles. To confirm their tertiary structure and oligomeric status 4-12% gradient Blue Native PAGE was run. The Nano A protein form an oligomer with expected molecular weight of approximately ˜140 kDa suggesting pentameric structure and Nano C with a molecular weight of approximately ˜800 kDa suggesting formation of higher order oligomers. The results are presented at FIG. 8.

### Example 9: Antigenicity of Nano A as Measure by ELISA

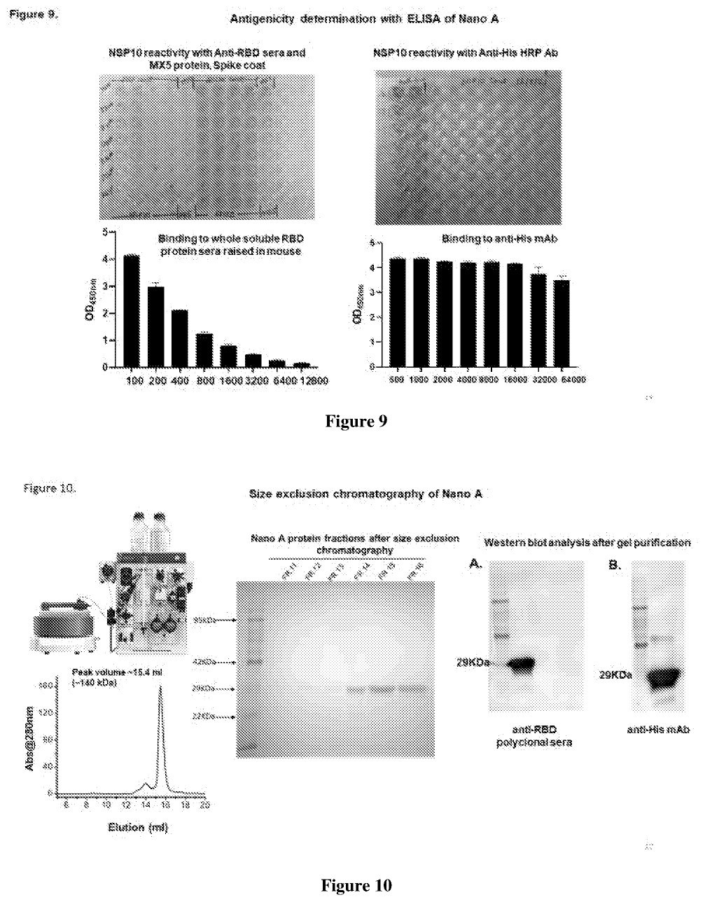

The ability of the Nano A proteins to bind to whole RBD soluble protein polyclonal mouse sera were measured by ELISA using Nano A protein coated at a concentration of 2 μg per well in the maxsorb 96 well plate in coating buffer incubated over night at 4° C. Antibody titers were calculated as the serum dilution giving OD450 nm readings after subtracting the background levels using prebled control serum at the same dilutions. As shown in FIG. 6, the nano A soluble protein binds to the whole RBD soluble protein at high titer confirming the presence of RBD peptides at the N terminal and to higher titer to anti-His antibody, thus suggesting binding to His tag present at the C terminal of the construct as shown in FIG. 9.

### Example 10: Size Exclusion Chromatography Purification of Nano A and Subsequent Biochemical Analysis

The Nano A protein purified by Ni-NTA column was concentrated and loaded on a Superdex 16/60 size exclusion chromatography column equilibrated with PBS. A single peak at elution volume of 15 ml eluted for the SEC column corresponded to a molecular weight of approximately ˜140 KDa. The identity of the eluted fraction was confirmed by SDS-Page analysis and Western blot using HRP conjugated anti-His antibody and polyclonal sera of anti-RBD. The results in FIG. 10 suggest the Nano A protein forms oligomeric self-assembled nano structure and there is minimal aggregation.

### Example 11: Secondary Structure Signature of Nano A (A) and Nano C (B) as Measured by Using Circular Dichroism (CD) Spectroscopy

The secondary structure and stability of the Nano proteins by CD spectra analysis was assessed. Far-UV CD spectra were acquired on a Jasco-815 spectropolarimeter. The concentration of the protein used is 5 μM. Cuvette of path length of 0.2 cm was used and spectra were collected from 260 to 190 nm at a rate of 100 nm/min and data pitch of 1 nm for each protein, with averaging of 10 scans for noise reduction. Contributions of the buffer to the spectra were electronically subtracted and for each spectrum, mean residual ellipticity (MRE) was calculated and plotted. The secondary structure signature of Nano A protein is showing peak minima at about 208 nm suggesting (FIG. 11A) suggesting it is predominantly β-sheet rich protein and small amount of α-helix as indicated by peak minima contribution at 222 nm. The Nano C protein is predominantly α-helix with peak minima at 222 nm (FIG. 11B).

### Example 12: Tag Free Purification of Nano A in Presence of TEV Protease and Subsequent Western Blot Analysis

For the ease of purification, the Nano constructs (Nano A, B and C) were expressed with 6×His preceding TEV protease site. The Ni-NTA affinity chromatography was applied as the first step of purification. Forever regulatory compliance it is necessary to remove the tag for further downstream application. To remove the 6×His-tag the Nano A purified protein was treated with TEV protease in PBS buffer for overnight at room temperature. The cleaved protein fraction was passed over NI-NTA column leading to the collection of His tag removed protein of interest in the flow through. The flow through fraction was collected and concentrated. To test the efficiency of removal of the His-tag the purified protein was run on a 12% SDS-PAGE and Western blot was performed with HRP conjugated anti-His antibody and anti-RBD polyclonal sera. Probing with HRP conjugated anti-His antibody confirms that treatment with TEV protease completely removes the His-tag.

### Example 13: Immunogenicity Assessment of Tagless Nano A Protein Immunogen (DS1)

BALB/c mice of 6-8 weeks of age were used for this study, which were inbred at the THSTI small animal facility. All experiments were made to minimize animal suffering and carried out in accordance with the principles of humanity described in the relevant Guidelines of the CPCSEA, the protocol was approved by the Institutional Animal Ethics Committee (IAEC Approval number: IAEC/THSTI/121). Animals were randomly divided into four groups (six mice per group for soluble immunogen and 3 mice per group for adjuvant+PBS control and mammalian expressed soluble whole RBD protein from delta variant). The first group A was immunized with the TEV cleaved Nano A protein 40 μg along with AddaVax™ adjuvant (InvivoGen, USA) in a 1:1 ratio, the second group B with 40 μg along with AddaVax™ adjuvant (InvivoGen, USA) in a 1:1 ratio, the third group C with adjuvant+PBS control and fourth group D were injected with 30 μg of soluble delta-RBD protein. The immunization dosing was scheduled as 0 day primer, first boost at 21 day and if required second boost at 42 day and mice were administered the immunogen intramuscularly. Serum samples are scheduled to collected prior to the first immunization (pre-immune) and 2 weeks after each immunization.

### Example 14: Humoral Responses of Nano A Primed Immune Sera

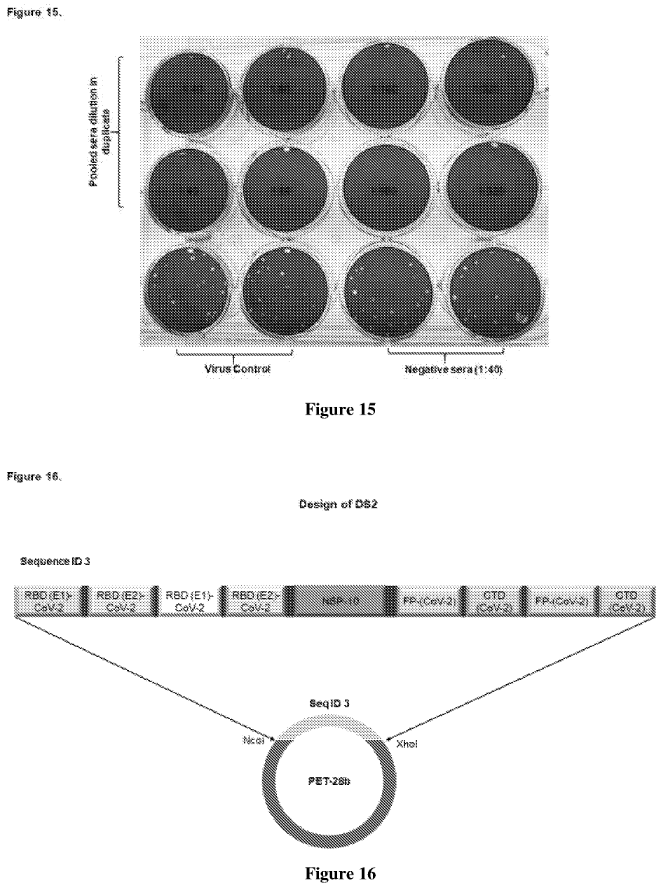

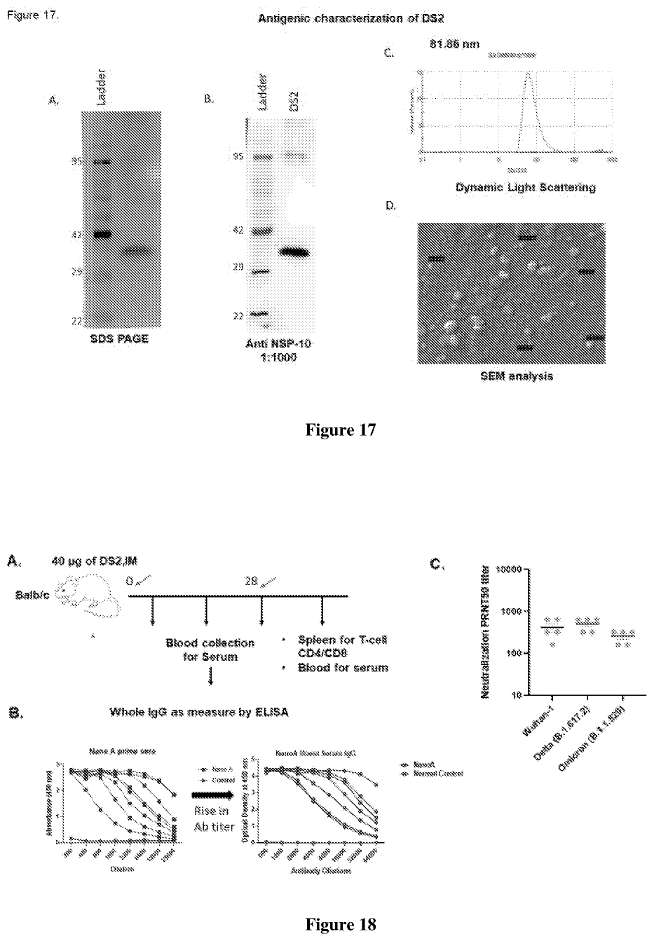

Binding antibody titers (IgG) were evaluated using ELISA plates coated with 100 μl/well of 1 mg/ml concentration of protein immunogens or peptides followed by incubation with serial diluted sera from animals immunized in group A and B after prime. A. Binding curves of diluted sera after the prime to Nano A homologous protein coated at a concentration of 1 mg/ml. B Binding curves of diluted sera after the prime to soluble whole wild type RBD protein from mammalian expression system (from wildtype SARS-COV-2 Wu-1 strain) coated at a concentration of 1 mg/ml. C. Binding curves of diluted sera after the prime to soluble RBD protein from mammalian expression system from SARS-COV-2 B.1.617.2 delta strain coated at a concentration of 1 mg/ml. D. Binding curves of diluted sera after the prime to RBD peptide. E. Binding curves of diluted sera after the prime to CTD or HR 2 peptide. Values plotted are the end point titers in duplicate generated in six mice per group. Statistical significance was determined using the one-way ANOVA test (p<0.05). The group C and D did not show significant results (Data not shown). FIG. 15 depicts neutralization titer of immunized boost sera of tagless Nano A (DS1) against SARS-COV-2 Wu-1 live virus as measured by plaque neutralization assay.

### Example 15: Development and Design of DS2

FIG. 15 (A) shows stapled SARS-COV-2 spike protein 20 mer RBD peptide from SARS-COV-2 Delta variant (sequence E1 and sequence E2) alternately stapled in N terminal of nano cage NSP10 and conserved 20 mer peptides from the fusion peptide and 20-mer C terminal domain from SARS-COV-2 ancestral Wuhan-1 strain by linkers to the C terminal domain of NSP-10. These stapled peptides or epitopes are incorporated into NSP-10 protein in both N terminus consisting of stapled RBD peptides or epitopes and C terminus consisting of stapled epitopes followed by stop codon (without any tag). FIG. 15 (B) depicts plasmid map (Expression Vector) of DS2.

### Example 16 Antigenic Characterization of DS2

FIG. 17A represents the purified proteins of DS2 as shown in 12% SDS-PAGE gel of approximate molecular weight of 39 kDa. The sequence of the DS2 consists of 363 amino acids of precited molecular weight of ˜39 kDa. FIG. 17 B represents western blot analysis of the purified protein probed by anti-mouse NSP10 polyclonal antibodies used as primary antibodies. FIG. 17 C represents the size of the DS2 protein as measured by dynamic light scattering (DLS) ˜81 nm and the proteins are found to be homogenous. FIG. 17 D represents that the shape of the synthesized DS2 protein was found to be circular with irregular surface as evaluated by Scanning electron microscopy (SEM). As far as size is concerned it was depicted in the range of 82-158 nm.

### Example 17 Immunogenicity Assessment of the Immunogen DS2 in BALB/c Mouse

BALB/c mice of 6-8 weeks of age were used for this study, which were inbred at the THSTI small animal facility. Animals were immunized with the tagless DS2 antigen 40μg along with AddaVax™ adjuvant (InvivoGen, USA) in a 1:1 ratio intramuscularly, one prime-one boost approach at 28 days interval. The blood was collected at 14 days post immunization for assessment of whole IgG binding ELISA. The spleen was collected 14 days after the boost to assess the cellular responses. The neutralization titer of immunized boost sera of tagless DS2 against SARS-COV-2 Wu-1. Delta and Omicron live viruses as measured by plaque neutralization assay.

### Example 18: Designing and 3D Model of the Immunogen DS3

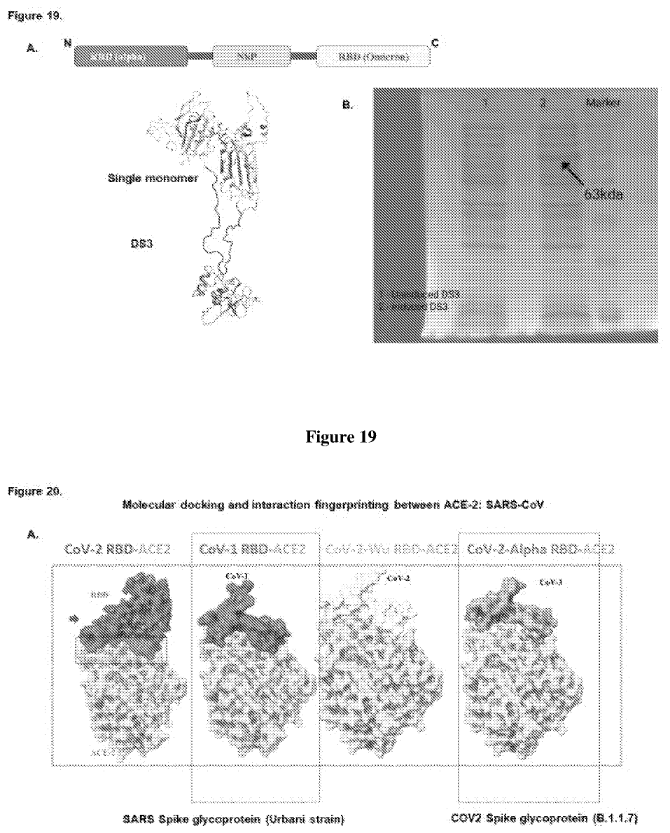

FIG. 19A illustrates designing of immunogenDS3 which consists of RBD domain of SARS-COV-2 Alpha variant in N terminal domain of the NSP10 and RBD domain of Omicron variant in the C terminal domain. FIG. 19B represents the expression of the DS3 antigen. The immunogen DS3 is purified using routine techniques.

### Example 19: Bioinformatic Analysis of SARS-COV-1, CoV-2 and MERS Spike Domains for Designing of Betacorona Vaccine

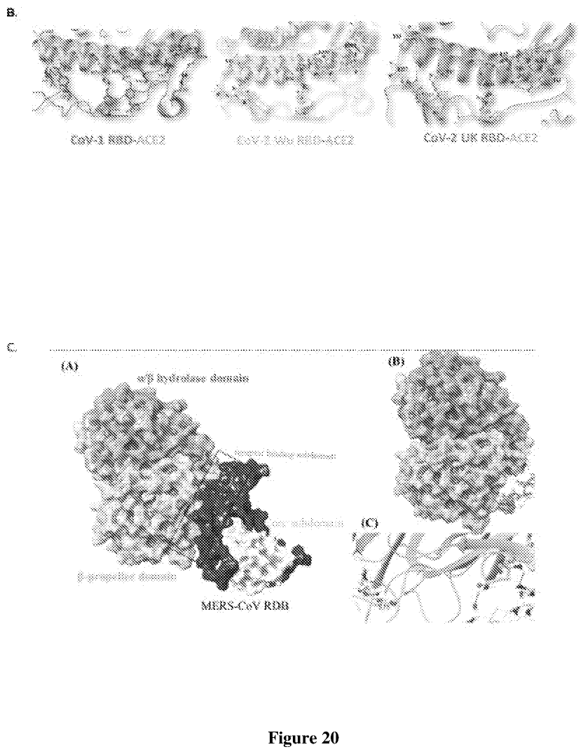

Bioinformatic analysis of SARS-COV-1, CoV-2 and MERS spike domains were performed for designing of betacorona vaccine. FIG. 20A shows the molecular docking and interaction fingerprinting between ACE-2 receptor and the RBD as shown for SARS-COV-1, SARS-COV-2 ancestral Wuhan-1 RBD and SARS-COV-2 Alpha RBD and ACE2 receptor. The interface interacting residue between the ACE and peptides are shown in ball and stick representation in FIG. 20B. The Hydrogens are highlighted in red and salt bridge are shown in orange color. FIG. 20 C shows molecular docking and interaction fingerprinting of Dipeptidyl peptidase-4 receptor for MERS. FIG. 20 C-A shows DPP4 extracellular domain consist of N-terminal β-propeller domain (mauve) and c-term alpha β-hydrolase (orange). FIG. 20 C-B shows MERS-COV RBSD contain a core subdomain (yellow color) and receptor binding subdomain (blue color) surface view of the predicted binding pose for peptide with DPP4. FIG. 20 C-C shows the interacting residue of the interface in ball and stick representation.

### Example 20: Designing of the Immunogen Pan-N3

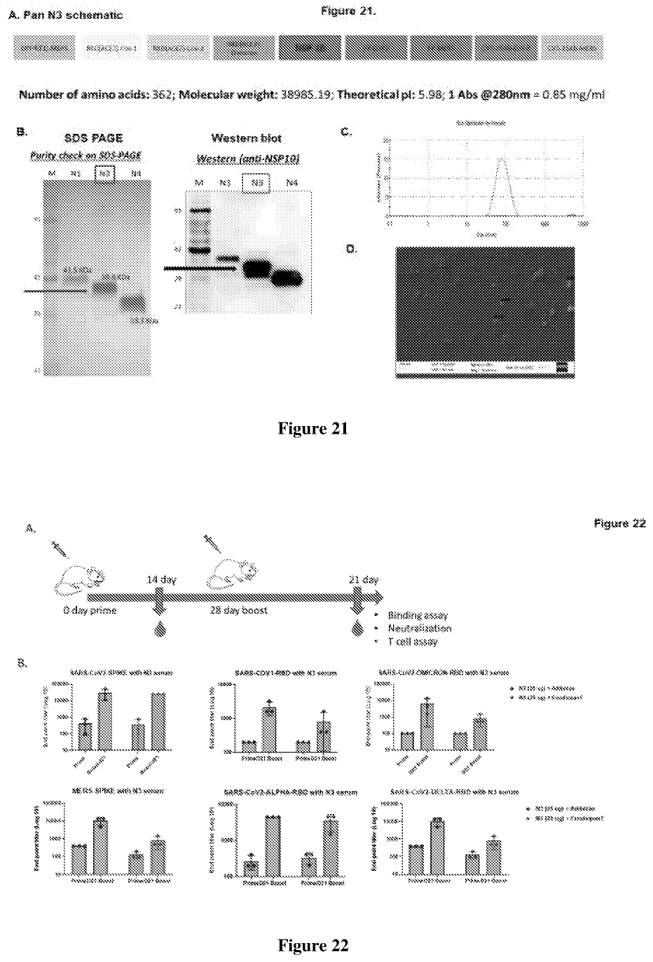

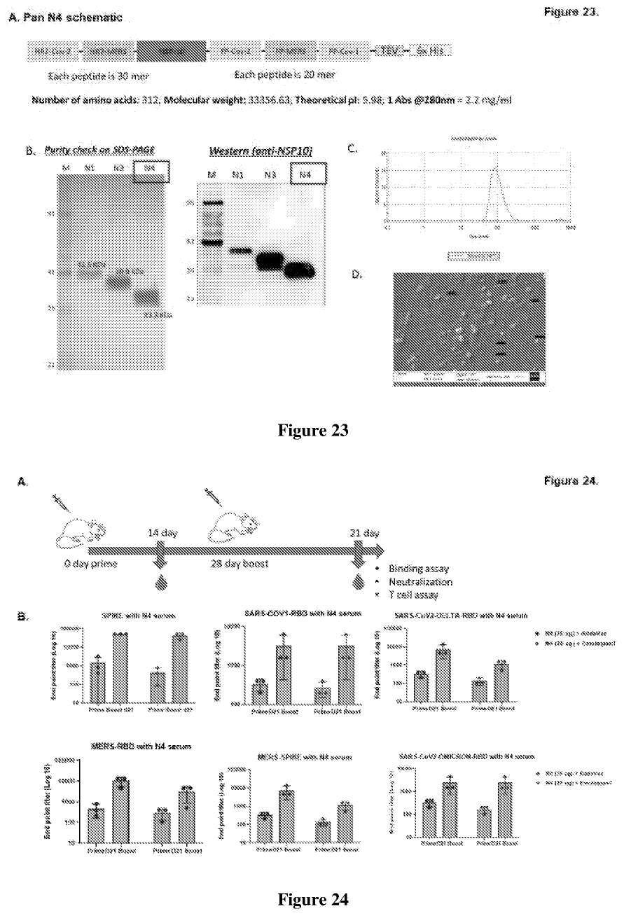

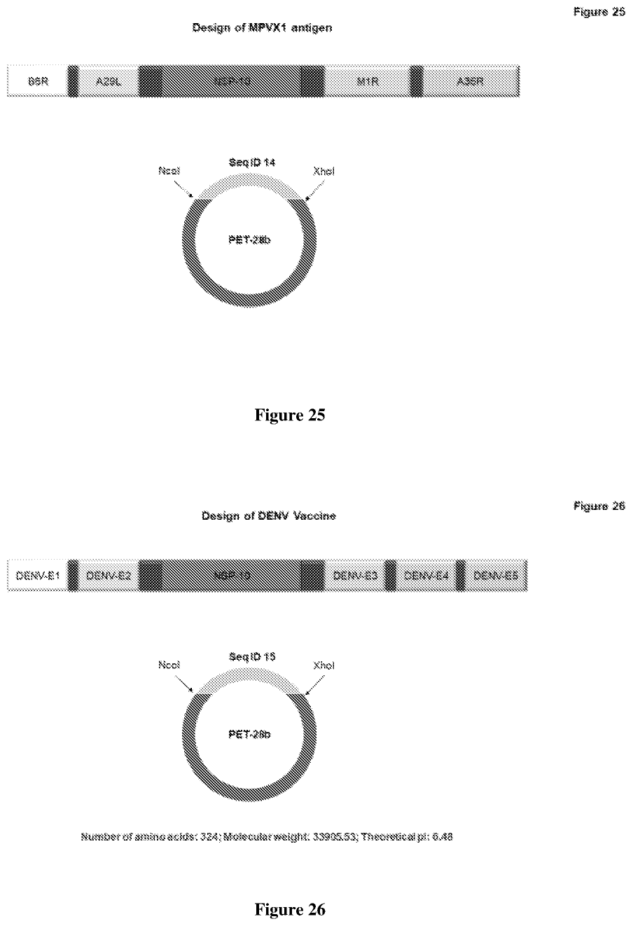

FIG. 21 shows the designing of the immunogen Pan-N3, which consists of epitopes from small epitopes (20 mers) region from receptor binding site for each of MERS, CoV-1, CoV-2 and Omicron at the N-terminal of NSP-10 nano cage; C-terminal has fusion peptide from heptad repeat2 domain for each CoV-2, CoV-1 and MERS and conserved neutralizing linear epitope from antibody CV3-25. FIG. 21B further represents the antigenic characterization.

### Example 21: Immunogenicity Assessment of the Immunogen pan-N3 in BALB/c Mouse

BALB/c mice of 6-8 weeks of age were used for this study, which were inbred at the THSTI small animal facility. Animals were immunized with the pan-N3 antigen 25 μg along with AddaVax™ and Emulsipan adjuvant in a 1:1 ratio intramuscularly, one prime-one boost approach at 28 days interval. The blood was collected at 14 days post immunization for assessment of whole IgG binding ELISA. The spleen was collected 14 days after the boost to assess the cellular responses.

### Example 22: Designing of the Immunogen pan-N4

FIG. 23A shows pan-N4 consists of 30 mer conserved epitopes from heptad repeat 2 of MERS an CoV-2 at the N-terminal (this epitope is highly conserved with SARS-COV-1, CoV-2 and variants of concerns) fused to NSP10; the C-terminal has fusion peptide from heptad repeat2 domain for each CoV-2, CoV-1 and MERS which are 20 mer long. FIG. 23 B. represents the antigenic characterization of the immunogen pan-N4.

### Example 23: Immunogenicity Assessment of the Immunogen pan-N4 in BALB/c Mouse

BALB/c mice of 6-8 weeks of age were used for this study, which were inbred at the THSTI small animal facility. Animals were immunized with the pan-N3 antigen 25 μg along with AddaVax™ and Emulsipan adjuvant in a 1:1 ratio intramuscularly, one prime-one boost approach at 28 days interval. The blood was collected at 14 days post immunization for assessment of whole IgG binding ELISA. The spleen was collected 14 days after the boost to assess the cellular responses.

### Example 24: Designing of Subunit Immunogen Against the Emerging Monkey Pox Virus

Antigens B6R and A29L are attached to the N terminal domain of the NSP10 and the antigens M1R and A35R are stapled to the C terminal domain of the NSP10by using linkers. The whole polypeptide was codon optimized as describe above and was cloned into the pET28a vector for expression in Escherichia coli. The cloned plasmids were propagated in the DH5-alpha strain of E. coli. For protein expression the plasmid with the sequence of interest were transformed in Rosetta (DE3) strain of E. coli. After transformation, the Rosetta (DE3) strain of E. coli. carrying the plasmid was grown in the super broth medium and overexpression of protein was induced at OD of 0.6 using 0.5-1.0 mM and further cultured at 18° C. for overnight or 37° C. for 4 hrs.

### Example 25: Designing of Subunit Immunogen Against the Dengue Virus (DENV)

Here the conserved B cell epitopes from Dengue virus serotypes DENV 2 are linked to the N terminal domain of NSP10 and three epitopes to the C-terminal domain of the NSP10 by using linkers. The whole polypeptide was codon optimized as describe above and was cloned into the pET28a vector for expression in Escherichia coli. The cloned plasmids were propagated in the DH5-alpha strain of E. coli. For protein expression the plasmid with the sequence of interest were transformed in Rosetta (DE3) strain of E. coli. After transformation, the Rosetta (DE3) strain of E. coli. carrying the plasmid was grown in the super broth medium and overexpression of protein was induced at OD of 0.6 using 0.5-1.0 mM and further cultured at 18° C. for overnight or 37° C. for 4 hrs.

1. A method of designing a multivalent self-assembled nanoparticle vaccine comprising the steps of:
i. identifying an antigenic peptide or peptide selected from one or more immunogenic peptides, epitopes, domains from the conserved or variable region of a virus or different strains of a virus of the same family;
ii. screening one or more antigenic peptide, immunogenic peptides, epitopes or domains identified in step (i) and selection of at least one immunogenic peptide or epitope or domain or antigenic peptide showing high immunogenicity;
iii. stapling said one or more antigenic peptide, immunogenic peptide, epitope or domain screened in step (ii) by one or more linker;
iv. stitching said stapled one or more antigenic peptide, immunogenic peptide, epitope or domain of step (iii) to a nano-cage by one or more linker to obtain a multipeptide or multi epitopes or multidomains;
v. optimizing codons for expressing said stitched multipeptides or multidomains or multi epitopes to obtain an encoding polynucleotide;
vi. developing an expression vector comprising said encoding polynucleotide;
vii. expressing said vector in a cell to obtain a multivalent self-assembled nanoparticle vaccine.

2. The method as claimed in claim 1, wherein the immunogenic peptides are B cell epitopes, immunogenic or antigenic epitopes selected from the virus structural envelope protein, nucleoprotein (NP), Matrix 1 protein (Ml), Matrix 2 protein (Ml), non-structural protein (NS); or T cell epitopes selected from enveloped viral proteins or fragments, spike or envelop protein, nucleoprotein (NP), Matrix 1 protein (Ml), Matrix 2 protein (M2), non-structural protein (NSP); and virions, antigenic peptides selected from intra cellular (IMV) and extracellular virions (EEVs) structural proteins.

3. The method as claimed in claim 1, wherein the antigenic peptide, epitope or domain is selected from SARS-COV-2 spike domain from ancestral Wuhan-1 strain, epitopes from spike domain of SARS-COV-2 variants of concern (VoC), SARS-CoV-1, MERS; other Corona viruses; epitopes from monkey pox, epitopes from Dengue serotype 2.

4. The method as claimed in claim 1, wherein the linker is selected from glycine rich linkers, alanine-rich linker, five-amino acid Gly linker linker, and the like.

5. The method as claimed in claim 4, wherein the linker is Gly-Gly-Ser-Gly or Gly-Ser-Ser-Gly.

6. The method as claimed in claim 1, wherein the nano-cage is selected from non-structural protein 10 (nsp10), Ferritin and Lumazine.

7. The method as claimed in claim 1, wherein the cell is a prokaryotic cell.

8. The method as claimed in claim 1, wherein the method further comprises the step of flanking the C-terminal of the multipeptide or multiepitope before step (v) with Tobacco Etch Virus (TEV) protease and a tag selected from His-tag, ubiquitin tag, NusA tag, chitin binding domain, green fluorescent protein (GFP), hemagglutinin influenza virus (HAG), glutafhione-Stransferase (GST), streptococcal protein G, staphylococcal protein A, T7genel0,avidin/streptavidin/Strep-tag, trpE, chloramphenicol acetyltransferase, lacZ (b-Galactosidase), His-patch thioredoxin, thioredoxin, FLAG™ peptide (Sigma-Aldrich), S-tag, and T7-tag.

9. The method as claimed in claim 1, wherein the expression vector is selected from a plasmid, phages, virus DNA, or combinations thereof.

10. The method as claimed in claim 9, wherein the plasmid is pBR 322, pUC, pGEM, and pBluescript.

11. The method as claimed in claim 9, wherein the phage is lambda-gt-Wes, Charon 28, Ml 3 derived phages.

12. The method as claimed in claim 9, wherein the virus DNA is SV40, adenovirus or polyoma virus DNA.

13. The method as claimed in claim 2, wherein the immunogenic peptides are SARS-COV-2 receptor binding domain (RBD) peptides, two or more peptides from heptad repeat (2) region from spike protein of SARS-COV-2 virus, SARS-COV-2 spike domain from ancestral Wuhan-1 strain, epitopes or RBD from spike domain of SARS-COV-2 VoC, SARS-COV-1, MERS, antigens B6R, A29L, MIR and A35R from Monkey pox virus, epitopes from DENV 2 serotype.

14. A multiepitope self-assembled nanoparticle vaccine comprising
i. a multi-peptide or multi epitopes or multi-domains consisting of or one or more immunogenic peptides stitched by one or more linkers; and
ii. a nanocage,
wherein the multi-peptide or multi epitope or multi-domains is stapled with the nanocage.

15. An engineered SARS-COV-2 multipeptide or multiepitope or multidomain having SEQ ID: 5, comprising at least two SARS-COV-2 receptor binding domain (RBD) peptides of SEQ ID: 1 and at least two peptides from heptad repeat (2) region from spike protein of SEQ ID: 2 linked by linker of SEQ ID: 3, and nanocage of SEQ ID: 4.

16. A polynucleotide encoding the SARS-COV-2 multipeptide having SEQ ID: 5.

17. An engineered SARS-COV-2 multipeptide having SEQ ID: 7, comprising at least two SARS-COV-2 receptor binding domain (RBD) peptides of SEQ ID: 1 linked by linker of SEQ ID: 3, and nanocage of SEQ ID: 6.

18. A polynucleotide encoding the SARS-COV-2 multipeptide having SEQ ID: 7.

19. An engineered SARS-COV-2 multipeptide having SEQ ID: 8, comprising at least two peptides from heptad repeat (2) region from spike protein of SEQ ID: 2 linked by linker of SEQ ID: 3, and nanocage of SEQ ID: 6.

20. A polynucleotide encoding the SARS-COV-2 multipeptide having SEQ ID: 8.

21. A multiepitope self-assembled nanoparticle vaccine having SEQ ID: 9.

22. An engineered SARS-COV-2 multivalent immunogen DS2 having SEQ ID: 10, comprising epitopes from SARS-COV-2 ancestral Wu-1 and epitopes from the SARS-COV-2 Delta variant.

23. An engineered SARS-COV-2 multivalent immunogen DS3 having SEQ ID 11 comprising the RBD domain from SARS-COV-2 variant Alpha in the N-terminal domain of NSP-10 and RBD domain from the SARS-COV-2 variant Omicron in the C terminal domain of NSP-10.

24. An engineered pan beta corona vaccine pan-N3 having SEQ ID: 12 comprising epitopes from SARS-COV-2, SARS-COV-1 and MERS spike domains.

25. An engineered pan beta corona vaccine pan-N4 having SEQ ID: 13 comprising the conserved epitopes from SARS-COV-2, SARS-COV-1 and MERS spike domains.

26. An engineered Monkey pox virus vaccine MPXV-1 having SEQ ID 14 comprising antigens from both IMV and EEV particles.

27. An engineered Dengue virus vaccine DN-1 having SEQ 1D 15 comprising conserved epitopes from DENV2 serotype linked by linker, and nanocage.
